(19) United States
(12) Patent Application Publication
(10) Pub. No.: US 2023/0346912 A1
(43) Pub. Date: Nov. 2, 2023

(54) RESTRICTIVE EPITOPE PEPTIDE OF MAJOR HISTOCOMPATIBILITY COMPLEX B2 OF H9N2 SUBTYPE AVIAN INFLUENZA VIRUS AND APPLICATION THEREOF

(71) Applicant: South China Agricultural University, Guangzhou (CN)

(72) Inventors: Ming LIAO, Guangzhou (CN); Manman DAI, Guangzhou (CN); Yusheng JIA, Guangzhou (CN)

(21) Appl. No.: 18/305,735

(22) Filed: Apr. 24, 2023

(30) Foreign Application Priority Data
Apr. 27, 2022 (CN) 202210449829.4

(51) Int. Cl. A61K 39/145; C07K 14/005; A61P 31/16

(52) U.S. Cl. CPC A61K 39/145; C07K 14/005; A61P 31/16

(57) ABSTRACT

Disclosed are a restrictive epitope peptide of a major histocompatibility complex (MHC) B2 of an H9N2 subtype Avian influenza virus (AIV) and an application thereof, and relate to the technical field of genetic engineering. The restrictive epitope peptide has an amino acid sequence as shown in SEQ ID NO.1, SEQ ID NO.2, SEQ ID NO.3 or SEQ ID No.4.

### CROSS-REFERENCE TO RELATED APPLICATIONS

This application claims priority to Chinese Patent Application No. 202210449829.4, filed on Apr. 27, 2022, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 347_004_2023_3574 sequence listing
Creation date: Apr. 20, 2023
Byte size: 427,819 bytes

### TECHNICAL FIELD

The present application relates to the technical field of genetic engineering, and in particular to a restrictive epitope peptide of a major histocompatibility complex (MHC) B2 of an H9N2 subtype avian influenza virus (AIV) and an application thereof.

### BACKGROUND

Avian influenza virus (AIV) is a kind of segmented ribonucleic acid (RNA) virus belonging to the genus Influenza virus A of the family Orthomyxoviriade, with hosts ranged from various avian species to mammals including humans. Based on the serological differences in Hemagglutinin (HA) and Neuraminidase (NA), AIV can be classified into 18 HA subtypes and 11 NA subtypes, with H9N2 subtype AIV being prevalent in poultry in China. Despite of being a low pathogenic avian influenza, H9N2 subtype AIV can still cause significant economic losses by reducing egg production or co-infecting chickens with other pathogens in poultry, making it essential to strengthen prevention, control and research on H9N2 subtype AIV.

Currently, inactivated vaccines are mainly used for preventing and controlling this virus; however, the virus is likely to mutate and evade from the recognition of antibodies as a result of prolonged immune selection pressure, which leads to insufficient protection across subtypes by the specific antibodies produced by the vaccine. Therefore, the development of vaccines with broader coverage and longer-lasting protection is important for preventing and controlling avian influenza.

Extensive studies have shown that influenza-specific CD8⁺ T cells not only participate in viral clearance but also provide cross-protection against other subtypes of influenza viruses, as demonstrated by Dai et al. who found that CD8⁺ T cell response plays an important role in fighting AIV infection by comparing key protective factors generated by H9N2 AIV infection and vaccine immunization-induced immune response in pathogen-free chickens; while Seo et al. found that chickens infected with H9N2 AIV showed higher survival rates in H5N1 AIV infection assays and the survival rate of chickens infected with H5N1 AIV was increased by subsequent injection with activated H9N2 AIV-specific CD8⁺ T cells. In summary, the development of a vaccine that can induce T-cell immune responses is of great importance for the prevention and control of H9N2 AIV.

Immunogenic epitopes are prerequisites for inducting immune effects in T cells. By March 2022, the Immune Epitope Database (IEDB) showed a total of 34 T-cell epitopes of AIV against chickens, of which 24 T-cell epitopes had been functionally validated for immunogenicity, including 22 CD8⁺ T-cell epitopes and 2 CD4⁺ T-cell epitopes on nucleoproteins, polymerase proteins, matrix protein 1 and haemagglutinin, covering three subtypes of H5N1, H5N8 and H7N1, but no epitopes have been reported for the H9N2 subtype of AIV. Therefore, a systematic screening for immunogenic AIV epitopes is required to address the current long-term prevalence of H9N2 subtype AIV.

Antigenic epitopes are recognized by T cell receptors (TCRs) through binding to major histocompatibility complex (MHC) class I molecules; however, MHC class I molecules are polymorphic and different MHC class I molecules can bind different antigenic epitopes even for the same pathogen, so it is important to clarify the restriction of the MHC while screening for antigenic epitopes. Currently, chickens can be classified into 29 haplotypes from B1 to B29 based on the gene sequence of the MHC B gene region, of which haplotype B2, a common haplotype, has been widely reported to show resistance to certain diseases and is an excellent material for experimental studies on AIV and vaccine development.

### SUMMARY

The objectives of the present application are to provide a restrictive epitope peptide of a major histocompatibility complex (MHC) B2 of an H9N2 subtype avian influenza virus (AIV) and an application thereof, so as to solve the problems existing in the prior art. According to the present application, an animal model of H9N2 subtype AIV infection in B2 haplotype chickens is established to demonstrate the important role of cellular immune response in the resistance of B2 haplotype chickens to AIV infection, and potential epitopes in H9N2 subtype AIV viral proteins are systematically screened using the binding motif of B2 haplotype MHC class I molecular, and finally peptide epitopes with immunogenicity are identified through functional assays to facilitate the development of AIV epitope vaccines.

To achieve the above objectives, the present application provides technical schemes as follows:
the present application provides a restrictive epitope peptide of an MHC B2 of an H9N2 subtype AIV, where the restrictive epitope peptide has an amino acid sequence as shown in SEQ ID NO.1, SEQ ID NO.2, SEQ ID NO.3 or SEQ ID No.4.

The present application also provides an application of the restrictive epitope peptide in preparing vaccine against H9N2 subtype AIV.

The present application also provides a vaccine against H9N2 subtype AIV, including the restrictive epitope peptide.

The present application achieves the following technical effects:
according to the present application, firstly, H9N2 subtype AIV (A/Chicken/Hunan/HN/2015) strain is used to infect B2 haplotype (BW/G3) SPF chickens, and the success of the infection model is determined by examining cloacal virus shedding, oropharyngeal virus load, changes in T-cell subtypes in peripheral blood mononuclear cells (PBMC) and expression of immune-related genes in PBMC to act as a follow-up test material; then, candidate peptide epitopes that are potentially immunogenic against H9N2 subtype AIV are screened based on the motif (X-A/V/I/L/P/S/G-X-X-X-X-X-X-X-V/I/L) of B2 haplotype chicken MHC class I molecule identified in the National and Regional Joint Engineering Laboratory for Medicament of Zoonosis Prevention and Control, and the immunogenicity of the above peptides is finally verified by the ELISpot assay to identify valid T cell epitope.

### BRIEF DESCRIPTION OF THE DRAWINGS

In order to explain the embodiments of the present application or the technical schemes in the prior art more clearly, the drawings needed in the embodiments are briefly introduced below. Obviously, the drawings described below are only some embodiments of the present application, and other drawings are available according to these drawings without creative work for ordinary people in the field.

FIG. 1 shows viral load of oropharyngeal swabs of B2 haplotype chicken, with n=7.

FIG. 2 illustrates antibody levels in serum, with n=4.

FIG. 3 illustrates changes of CD8α⁺ T cells in PBMC of B2 haplotype chickens after infection, with n=4.

FIG. 4 illustrates changes of CD4⁺ T cells in PBMC of B2 haplotype chickens after infection, with n=4.

FIG. 5 illustrates changes of proportions of CD4⁺ and CD8α⁺ T cells in PBMC of B2 haplotype chickens after infection; n=4.

FIG. 6 shows changes of CD⁴⁺/CD8α+ T cells in PBMC of B2 haplotype chickens after infection, n=4.

FIG. 7 represents expressions of natural immune related genes in PBMC of B2 haplotype chickens after H9N2 avian influenza virus (AIV) infection.

FIG. 8 represents expressions of cytotoxic T cells (CTLs)-related genes in PBMC of B2 haplotype chickens after H9N2 AIV infection.

FIG. 9 represents expressions of Th2-related genes in PBMC of B2 haplotype chickens after H9N2 AIV infection.

FIG. 10A, FIG. 10B and FIG. 10C show expression levels of interferon gamma (IFN-γ) of the splenocytes which are stimulated with peptide pool, while FIG. 10A shows ELISpot results of pool_1-pool_28, FIG. 10B shows ELISpot results of pool_29-pool_56, and FIG. 10C shows ELISpot results of pool_57-pool_85, with n=3 except in positive control.

FIG. 11 shows representative images of IFN-γ ELISpot responses of #1 chicken.

FIG. 12 shows representative images of IFN-γ ELISpot responses of #2 chicken.

FIG. 13 shows representative images of IFN-γ ELISpot responses of #3 chicken.

FIG. 14 shows secretion levels of IFN-γ in spleen lymphocytes of the #1 chicken, with three technical replicates of each peptide, except for the positive control.

FIG. 15 shows secretion levels of IFN-γ in spleen lymphocytes of the #2 chicken, with three technical replicates of each peptide, except for the positive control.

FIG. 16 shows secretion levels of IFN-γ in spleen lymphocytes of the #3 chicken, with three technical replicates of each peptide, except for the positive control.

### DETAILED DESCRIPTION OF THE EMBODIMENTS

Various exemplary embodiments of the present application are now described in detail and this detailed description should not be considered as limiting the present application, but should be understood as a more detailed description of certain aspects, features and embodiments of the present application.

It should be understood that the terms described in the present application are intended to describe particular embodiments only and are not intended to limit the present application. Furthermore, with respect to the range of values in the present application, it is to be understood that each intermediate value between the upper and lower limits of the range is also specifically disclosed. Each smaller range between any stated value or intermediate value within a stated range, and any other stated value or intermediate value within a stated range is also included in the present application. The upper and lower limits of these smaller ranges may be independently included or excluded from the scope.

Unless otherwise stated, all technical and scientific terms used herein have the same meaning as is commonly understood by those of ordinary skill in the field described in the present application. Although the present application describes only preferred methods and materials, any methods and materials similar or equivalent to those described herein may also be used in the implementation or testing of the present application. All literature referred to in this specification is incorporated by reference for the purpose of disclosing and describing the methods and/or materials associated with said literature. In the event of conflict with any incorporated literature, the contents of this specification shall prevail.

Without departing from the scope or spirit of the present application, various improvements and variations can be made to specific embodiments of the specification of the present application, as will be apparent to those skilled in the art. Other embodiments derived from the specification of the present application are obvious to the skilled person. The specification and embodiments of the invention are only exemplary.

As used herein, the words “comprising”, “including”, “having”, “containing”, etc., are open-ended terms, i.e. meaning including but not limited to.

Terminology Explanation:

AIV: avian influenza virus; MHC I: class I major histocompatibility complex; ELISpot: enzyme-linked immune-absorbent spot; PBMC: peripheral blood mononuclear cells; SPF chicken: specific pathogens free chicken; CTL: cytotoxic T lymphocytes; IFN-γ: interferon-gamma; DPI: days post infection; EID₅₀: 50% embryo infective dose of chicken; FBS: fetal bovine serum; PMA+Ionomycin: phorbol myristoyl acetate and ionomycin.

### Embodiment 1

### 1. Experimental Materials

### 1.1 Experimental Animals and Viruses

The chickens used in this experiment are 4-week-old B2 haplotype SPF chickens (BW/G3) purchased from the National Poultry Laboratory Animal Resource Center; the H9N2 subtype AIV (A/Chicken/Hunan/HN/2015) strain is isolated and stored in the National and Regional Joint Engineering Laboratory for Medicament of Zoonosis Prevention and Control.

### 1.2 Main Experimental Reagents

Total RNA extraction kit is purchased from Jianshi Biotechnology Company; chicken peripheral blood lymphocyte separation liquid kit; chicken organ tissue mononuclear cell separation kit and red blood cell lysis buffer are purchased from Tianjin Haoyang Biological Manufacture Co., Ltd; ChamQ SYRB qPCR Master Mix are purchased from Nanjing Vazyme Biotech Co., Ltd.; Chicken IFN-γ ELISpot BASIC kit is purchased from Mabtech Company; flow antibodies including Anti-chicken CD3 antibody, Anti-chicken CD4 antibody, Anti-chicken CD8α antibody are purchased from Southern Biotech Company; PMA+Ionomycin and 3,3′,5,5′-tetramethylbenzidine (TMB) ELISpot chromogenic substrates are purchased from Dakewe Biotech Co., Ltd. of China; RPMI-1640 medium and Fetal Bovine Serum (FBS) Australian fetal bovine serum are purchased from GIBCO Company of the United States.

### 1.3 Preparation of Main Solution

(1) 1640 complete medium: 45 milliliters (mL) RPMI-1640 medium, 5 mL inactivated FBS and 500 microliters (μL) streptomycin (100×) are added into a 50 mL centrifuge tube, and mixed evenly at 4° C.;

(2) flow buffer: 49 mL RPMI-1640 culture medium and 1 mL of inactivated FBS are added into a 50 mL centrifuge tube, and mixed evenly at 4° C.;

(3) cells cryopreservation solution: 45 mL inactivated FBS and 5 mL dimethylsulfoxide (DMSO) are added into a 50 mL centrifuge tube, and mixed evenly at 4° C.

### 2 Experimental Methods

### 2.1 Virus Propagation

H9N2 AIV is melted and diluted 1,000 times in sterile PBS to obtain diluted virus solution, 9-11-day SPF chicken embryos are sterilized and placed on the biosafety cabinets, with each chick embryo allantoic cavity inoculated with 100 μL diluted virus solution, followed by sealing and continuing culture of the chick embryo for 24 hours (h); the survival of the chicken embryos is observed 24 h after the inoculation and the dead embryos are discarded; the remaining live embryos are continued to be cultured for 72 h to collect the virus, followed by aspirating the allantoic liquid into a centrifuge tube with a pipette, centrifuging the liquid at 4 degrees Celsius (° C.) for 10 minutes (min) at 2,000 revolutions per minute (rpm) to obtain a supernatant, passing the supernatant through a 0.22 micrometers (μm) filter membrane and sub-packing, storing at −80° C. for later use.

### 2.2 Determination of Hemagglutination Titer

The titer is determined with reference to Chinese national standards of the latest edition (GB/T 18936-2020).

### 2.3 Determination of EID₅₀

The 50% embryo infective dose (EID₅₀) of chicken embryo is determined as follows:
the virus solution after propagation is taken out and melted on ice, then diluted 10 times with PBS; the virus solution with a dilution of 10⁻⁴-10⁻⁹ is used to inoculate chicken embryos according to the method of 2.1, with 5 chicken embryos at each dilution and followed by culture 72 h; then, 25 μL allantoic liquid is collected from each chick embryo, the hemagglutination titer is determined by the method of 2.2, and the EID₅₀ is calculated by Spearman-Karber method.

### 2.4 Establishment of Animal Model of H9N2 Subtype AIV infection in B2 Haplotype Chickens

H9N2 AIV virus solution is diluted with sterile PBS to 10⁷ EID₅₀/200 μL; the experimental animals are divided into two groups, including B2 haplotype chicken experimental group and B2 haplotype chicken control group, with 7 chickens in each group. The experimental group is challenged intranasally and intratracheally, where 200 μL of virus is injected in the eyes of each animal with one drop in the left and one drop in the right eye firstly, then the rest of the virus is injected through the nasal cavity of one side; the control group is inoculated with equal volume of PBS in the same way. Swabs from the oropharyngeal and cloaca of chickens, peripheral anticoagulation and non-anticoagulation are collected and detected on 3, 5, 7, 9 and 11 DPI.

### 2.4.1 Detection of H9N2 AIV Shedding of Infected Chickens

The swabs collected on the sampling days are stored at −80° C. for unified detection. After the swab is taken out, it is melted on the ice, fully vortexed, and then the impurities are removed by centrifugation at 4° C. and 12,000 rpm for 5 min; then the supernatant is filtered through a 0.22 μm filter, and the EID₅₀ is determined according to 2.3, usually with a dilution of 10⁰-10⁻⁶, so as to evaluate the virus shedding.

### 2.4.2 Detection of Serum Antibody Levels

The collected non-anticoagulants are placed at room temperature until the serum is precipitated, and the serum is collected into a 1.5 mL centrifuge tube, followed by centrifugation at 4° C. and 2,000 rpm for 10 min to remove the red blood cells, and the remaining serum is used to detect the serum antibody level.

The antibody level is detected by hemagglutination inhibition (HI) test, with reference to the latest edition of national standard (GB/T 18936-2020).

### 2.4.3 Detection of T Cell Percentage in Chicken PBMC

PBMC is separated according to the instruction of the kit, and appropriate cells are taken for flow staining; the following descriptions are based on 10⁶ cells per tube: cells are added in flow tubes, then 1 mL of flow Buffer is added, followed by centrifugation at 440 g for 6 min, and CD3, CD4 and CD8 antibodies are diluted in the dark according to the recommended concentrations in the instruction; the supernatant is discarded after centrifugation, with 100 μL of diluted antibody added to each tube for re-suspension, and incubated for 30 min at 4° C. in the dark; then, 1 mL Buffer is added, and centrifuged at 440 g for 6 min, with precipitate resuspended with 250 μL flow Buffer; the data are collected by an up-flow cytometry and analyzed by a FlowJo software.

### 2.4.4 Expressions of Immune-Related Genes in Chicken PBMC by Fluorescence Quantitative Polymerase Chain Reaction (PCR) Detection

The total RNA is extracted according to the total RNA extraction kit from Jianshi Biotechnology Company, briefly, taking an appropriate amount of cells, centrifuging at 440 g for 6 min, discarding the supernatant, adding 1 mL of TRIzol then whirling; then adding equal volume of anhydrous ethanol, mixing well, transferring the liquid to a No.2 column, centrifuging for 1 min, and removing the filtrate; adding 400 μL RNA washing solution 2 to the column, centrifuging for 1 min, and discarding the filtrate; add 80 μL of DNase I reaction solution to the column, reacting at room temperature for 15 min, then adding 400 μL of RNA washing solution 1, centrifuging for 1 min, and discarding the filtrate; placing for 2 min, transferring the No.2 column to a centrifugal tube without RNase, adding 50 μL RNase-free water preheated to 70° C. in advance, standing for 2 min, centrifuging for 1 min to elute RNA, and detecting the concentration with ultra-micro spectrophotometer, and storing at −80° C.

RNA is reverse-transcribed according to the system in Table 1 following a reverse transcription procedure as follows: performing reverse transcription PCR at 37° C. for 15 min, inactivating at 85° C. for 5 s, and storing at 4° C.

TABLE 1
Reverse Transcription System

| Components | Dosage |
|---|---|
| 5 × M-MLV-Mix | 4 μL |
| RNA | 10 μL (less than 1,000 ng) |
| Sterilized water | 6 μL |
| Total volume | 20 μL |

The reverse-transcribed complementary deoxyribonucleic acid (cDNA) is amplified by fluorescence quantitative PCR to detect the changes of immune-related genes in PBMC; the target genes and primers detected are as shown in Table 2, and the system is shown in Table 3. Reaction procedure: pre-denaturation at 95° C. for 30 s; cyclic reaction at 95° C., 10 s, 60° C. and 30 s, a total of 40 cycles; dissolution curve analysis 95° C., 15 s, 60° C., 60 s, 95° C., 15 s. The results are analyzed by 2^{-ΔΔCt} method with glyceraldehyde 3-phosphate dehydrogenase (GAPDH) gene as internal reference.

TABLE 2
qPCR25 Primers for Immune Related Genes

| Gene name | | Primer sequence (5′-3′) | Sequence ID |
|---|---|---|---|
| GAPDH | F-Primer | GAACATCATCCCAGCGTCCA | SEQ ID NO. 5 |
| | R-Primer | CGGCAGGTCAGGTCAACAAC | SEQ ID NO. 6 |
| Granzyme | F-Primer | CGGGAAGCAACTGTTGAAAT | SEQ ID NO. 7 |
| K | R-Primer | GAGTCTCCCTTGCAAGCATC | SEQ ID NO. 8 |
| Perforin | F-Primer | ATGGCGCAGGTGACAGTGA | SEQ ID NO. 9 |
| | R-Primer | TGGCCTGCACCGGTAATTC | SEQ ID NO. 10 |
| IFN-γ | F-Primer | CCTCCAACACCTCTTCAACATG | SEQ ID NO. 11 |
| | R-Primer | TGGCGTGCGGTCAAT | SEQ ID NO. 12 |
| TNF-α | F-Primer | GCTGTTCTATGACCGCCCAGTT | SEQ ID NO. 13 |
| | R-Primer | AACAACCAGCTATGCACCCCA | SEQ ID NO. 14 |
| IL-1β | F-Primer | GGTCAACATCGCCACCTACA | SEQ ID NO. 15 |
| | R-Primer | CATACGAGATGGAAACCAGCAA | SEQ ID NO. 16 |
| IL-2 | F-Primer | GCTAATGACTACAGCTTATGGAGCA | SEQ ID NO. 17 |
| | R-Primer | TGGGTCTCAGTTGGTGTGTAGAG | SEQ ID NO. 18 |
| NK lysin | F-Primer | GATGGTTCAGCTGCGTGGGATGC | SEQ ID NO. 19 |
| | R-Primer | CTGCCGGAGCTTCTTCAACA | SEQ ID NO. 20 |
| HMG-2 | F-Primer | AGAGCACAAGAAGAAGCAC | SEQ ID NO. 21 |
| | R-Primer | GTCTTTTAGGAGCGTTGGGGTC | SEQ ID NO. 22 |
| MHC-I | F-Primer | AAGAAGGGGAAGGGCTACAA | SEQ ID NO. 23 |
| | R-Primer | AAGCAGTGCAGGCAAAGAAT | SEQ ID NO. 24 |
| IFN-α | F-Primer | GACAGCCAACGCCAAAGC | SEQ ID NO. 25 |
| | R-Primer | GTCGCTGCTGTCCAAGCATT | SEQ ID NO. 26 |
| IFN-β | F-Primer | GCCCACACACTCCAAAACACTG | SEQ ID NO. 27 |
| | R-Primer | TTGATGCTGAGGTGAGCGTTG | SEQ ID NO. 28 |
| IL-6 | F-Primer | AAATCCCTCCTCGCCAATCT | SEQ ID NO. 29 |
| | R-Primer | CCCTCACGGTCTTCTCCATAAA | SEQ ID NO. 30 |
| IL-10 | F-Primer | AGCAGATCAAGGAGACGTTC | SEQ ID NO. 31 |
| | R-Primer | ATCAGCAGGTACTCCTCGAT | SEQ ID NO. 32 |
| TLR3 | F-Primer | ACAATGGCAGATTGTAGTCACCT | SEQ ID NO. 33 |
| | R-Primer | GCACAATCCTGGTTTCAGTTTAG | SEQ ID NO. 34 |
| TLR7 | F-Primer | TCTGGACTTCTCTAACAACA | SEQ ID NO. 35 |
| | R-Primer | AATCTCATTCTCATTCATCATCA | SEQ ID NO. 36 |
| MHC-I | F-Primer | AAGAAGGGGAAGGGCTACAA | SEQ ID NO. 37 |
| | R-Primer | AAGCAGTGCAGGCAAAGAAT | SEQ ID NO. 38 |
| MHC-II | F-Primer | CTCGAGGTCATGATCAGCAA | SEQ ID NO. 39 |
| | R-Primer | TGTAAACGTCTCCCCTTTGG | SEQ ID NO. 40 |
| IL-4 | F-Primer | TCGAGGAGTGACGGGTG | SEQ ID NO. 41 |
| | R-Primer | ACTATCCGGATGCTCTCCATC | SEQ ID NO. 42 |
| IL-5 | F-Primer | GGAACGGCACTGTTGAAAAATAA | SEQ ID NO. 43 |
| | R-Primer | TTCTCCCTCTCCTGTCAGTTGTG | SEQ ID NO. 44 |
| IL-13 | F-Primer | CTGCCCTTGCTCTCCTCTGT | SEQ ID NO. 45 |
| | R-Primer | CCTGCACTCCTCTGTTGAGCTT | SEQ ID NO. 46 |
| Granzyme | F-Primer | ACTCATGTCGAGGGGATTCA | SEQ ID NO. 47 |
| A | R-Primer | TGTAGACACCAGGACCACCA | SEQ ID NO. 48 |
| MDA5 | F-Primer | GGACGACCACGATCTCTGTGT | SEQ ID NO. 49 |
| | R-Primer | CACCTGTCTGGTCTGCATGTTATC | SEQ ID NO. 50 |
| CXCLi1 | F-Primer | AACTCCGATGCCAGTG | SEQ ID NO. 51 |
| | R-Primer | TTGGTGTCTGCCTTGT | SEQ ID NO. 52 |
| CXCLi2 | F-Primer | CATCATGAAGCATTCCATCT | SEQ ID NO. 53 |
| | R-Primer | CTTCCAAGGGATCTTCATTT | SEQ ID NO. 54 |
| TGF-β3 | F-Primer | TCTTTACATTGACTTCCGAC | SEQ ID NO. 55 |
| | R-Primer | TCCTCCCAACATAGTACAAG | SEQ ID NO. 56 |
| MX1 | F-Primer | AAGCCTGAGCATGAGCAGAA | SEQ ID NO. 57 |
| | R-Primer | TCTCAGGCTGTCAACAAGATCAA | SEQ ID NO. 58 |
| OASL | F-Primer | AGATGTTGAAGCCGAAGTACCC | SEQ ID NO. 59 |
| | R-Primer | CTGAAGTCCTCCCTGCCTGT | SEQ ID NO. 60 |
| ISG12-2 | F-Primer | TCAATGGGTGGCAAAGGAG | SEQ ID NO. 61 |
| | R-Primer | TACAGGGAGAGCAAAGAAGAGAAGA | SEQ ID NO. 62 |
| IFIT5 | F-Primer | CAGAATTTAATGCCGGCTATGC | SEQ ID NO. 63 |
| | R-Primer | TGCAAGTAAAGCCAAAAGATAAGTGT | SEQ ID NO. 64 |
| USP18 | F-Primer | CAACGTGGGAAGAGGAGAAA | SEQ ID NO. 65 |
| | R-Primer | ACTTCATGAGCGGAGAAGGA | SEQ ID NO. 66 |
| IRF3/7 | F-Primer | ACTGACCAGCCCAGGAACTCT | SEQ ID NO. 67 |
| | R-Primer | AAGGCTTTCCCAACCACAAA | SEQ ID NO. 68 |
| SST | F-Primer | GGTCCACGGTTATGGTGAAAG | SEQ ID NO. 69 |
| | R-Primer | GGTCAGAAATCACAACTCAAGCA | SEQ ID NO. 70 |
| KHSRP | F-Primer | CAGCGGGGAAATGATTAAGAAG | SEQ ID NO. 71 |
| | R-Primer | TTTGTGTGTGGGGATGGAGA | SEQ ID NO. 72 |
| PARP | F-Primer | ATTGTGGAGGAGCTGGGAGGAA | SEQ ID NO. 73 |
| | R-Primer | AGGCTTGCTGCACTTCCCATC | SEQ ID NO. 74 |

TABLE 3
Fluorescence Quantitative System

| Reagent | Volume | |
|---|---|---|
| 2 × ChamQ Universal | 10 | μL |
| SYBR qPCR Mix | | |
| F-Primer | 0.4 | μL |
| R-Primer | 0.4 | μL |
| cDNA | 2 | μL |
| ddH₂O | 7.2 | μL |

### 2.5 Screening Polypeptide Epitopes with Immunogenicity

According to the binding motif (X-A/V/I/L/P/S/G-X-X-X-X-X-X-V/I/L) of MHC class I molecular in B2 haplotype chickens determined in the National and Regional Joint Engineering Laboratory for Medicament of Zoonosis Prevention and Control, candidate polypeptide epitopes against H9N2 subtype AIV are screened, then the peptides are synthesized by Shanghai Top-Peptide Biotechnology Co., Ltd., with a purity of 95%, and each peptide is synthesized for 5 mg.

### 2.6 Immunogenicity Detection of Candidate Polypeptides

### 2.6.1 ELISpot Experiment for Detecting Immunogenicity of Peptide Pool

The synthesized polypeptide is dissolved in 200 μL DMSO, and then stored at −80° C.; five peptides are mixed into a pool, named from pool_1 to pool_85 respectively, and the immunogenicity is detected by ELISpot experiment, with experimental operation referring to the instructions of Chicken IFN-γ ELISpot BASIC Kit, the details are as follows:

the first day:
(1) the PVDF membrane in the cell wells is activated by 15 μL of 35% ethanol per well using a multichannel pipette for no more than 1 min; then each well is added with 200 μL sterile water for washing, and repeated for 4 times; the monoclonal antibody against chicken IFN-γ is diluted with sterile water at 1:33, and 100 μL is added to each well, and coated at 4° C. overnight;

the second day:
(2) the coating solution is discarded, and each well is washed with 200 μL sterile PBS for 4 times;
(3) each well is added with 200 μL 1640 complete culture medium and incubated at room temperature for 1-2 h;
(3) the solution is discarded, 100 μL spleen lymphocyte suspension (containing 3.5×10⁵ cells) is added to each well: at the same time, peptide library (final concentration of 10 micrograms per microliter (m/mL) for each peptide) is added to the experimental group, an equal volume of DMSO is added to the negative control group, and 10 μL of PMA+Ionomycin mixture from the Dakewe Biotech Co., Ltd. is added into the positive control group;
(4) after all the samples are added, the cell plate is placed into a 37° C. cell incubator containing 5% CO₂ for culture of at least 18 h;

the third day:
(5) after the culture, the culture medium and cells are discarded, and each well is washed five times by 200 μL sterile PBS; each well is added with 100 μL PBS containing 0.5% FBS and 1 μg/mL biotin-labeled detection antibody, and incubated for 2 h at room temperature; then the liquid is shaken off and 200 μL sterile PBS is added to each well for washing for 5 times;
(6) each well is added with 100 μL diluted streptavidin-labeled horseradish peroxidase (HRP) and incubated at room temperature for 1 h; then the liquid is discarded and each well is added with 200 μL sterile PBS for washing for 5 times;
(7) each well is added with 100 μL TMB chromogenic solution, and the reaction is stopped by washing the cell plate with ultrapure water until obvious spots appear at the bottom, then the cell plate is dried and counted in an automatic plate reader, and the number of spots is statistically analyzed.

### 2.6.2 ELISpot Experiment for Detecting Immunogenicity of Individual Peptide

The peptide pools in 2.6.1 that can significantly stimulate cells to produce spots are selected and detected for individual peptide by ELISpot experiment, the methods are the same as in 2.6.1.

### 2.7 Data Analysis

All the experimental data are statistically analyzed by using the software GraphPad Prism 8, in which ns means P>0.05, where the difference is not significant. * means P<0.05, with significant difference; ** means P<0.01, where the difference is extremely significant; *** means P<0.001, indicating an extremely significant difference, and **** means P<0.0001, where the difference is extremely significant.

### 3 Results

### 3.1 Detection of Detoxification of Infected Chicken

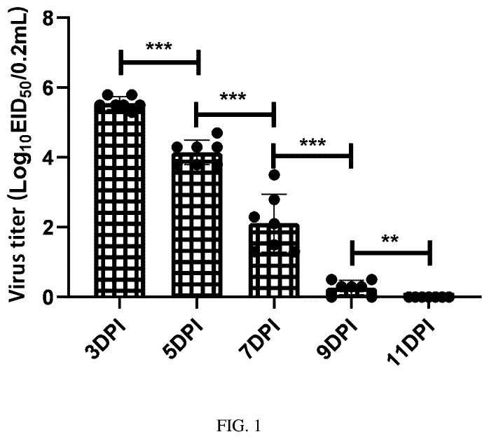

After H9N2 AIV infection of B2 haplotype chickens, swabs are collected and the virus shedding is detected according to 2.4.1. As shown in FIG. 1, in the H9N2 AIV-infected group, the viral load of oropharyngeal swabs peaked at 3 DPI, had fallen off since SDPI (p<0.0/), and disappeared at 11 DPI. The cloacal swabs were found to be positive at 3 DPI and SDPI and negative at 7 DPI in table 4, while control groups are all tested negative (the data is not included).

TABLE 4
The virus shedding of cloacal swabs in B2 Haplotype Chickens

| Groups | 3DPI | 5DPI | 7DPI |
|---|---|---|---|
| B2 chicken | 3.5, 4.3 (2/7) | 1.5, 1.5 (2/7) | (0/7) |

Note:
the figures outside brackets indicate the virus shedding of cloacal swabs of infected chickens, which is the value of log₁₀EID₅₀. The figures inside brackets/before brackets indicate the number of chickens that have been detoxified, and the figures after/indicate the total number of chickens.

### 3.2 Detection of Serum Antibody Level in Infected Chickens

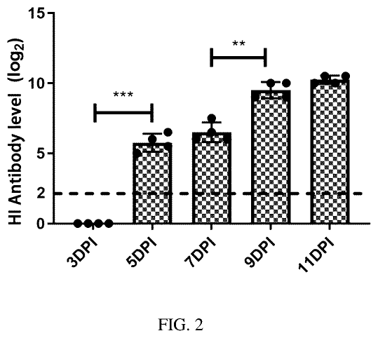

According to the results as shown in FIG. 2, the antibody levels are negative at 3 DPI (all less than 2 wells), all chickens tested at 5 DPI turn positive, and the HI antibody levels continue to rise until 11 DPI, indicating that the humoral immunity is initiated on 5 DPI, and the decrease the virus shedding in cloacal swabs from 5 DPI is associated with an increase in antibody levels. The control groups are all tested negative for antibody (the data is not included).

### 3.3 Changes of T Cell Subtypes in Chicken PBMC after Infection

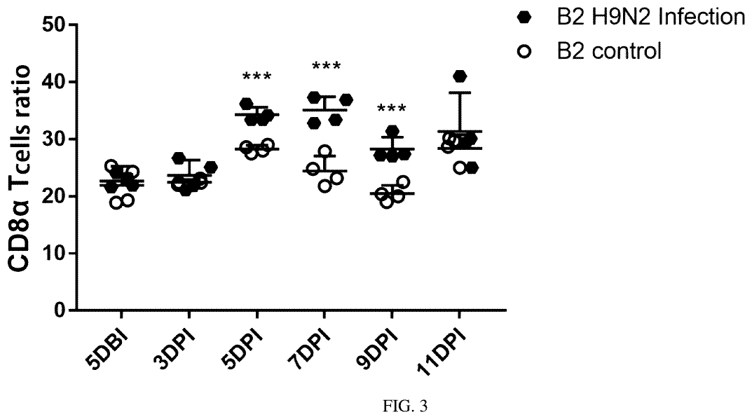

In order to detect the immune response of T cells in B2 haplotype chickens after challenge, peripheral blood of chickens is collected from jugular vein according to the experimental arrangement, and PBMC is isolated and stained with flow antibody. As can be seen from FIG. 3, the proportion of CD8⁺ T cells significantly increased in the H9N2 AIV-infected group compared to that in the control group at 5 DPI, 7 DPI and 9 DPI (P<0.001); the results show that significant CD8+ T cell proliferation is detectable in chicken PBMC from day 5 after H9N2 AIV infection of B2 haplotype chickens and persists until 9 DPI, suggesting that virus clearance from 5 DPI is associated not only with elevated antibody levels but also subjects to an important effect of CD8+ T cell immune response.

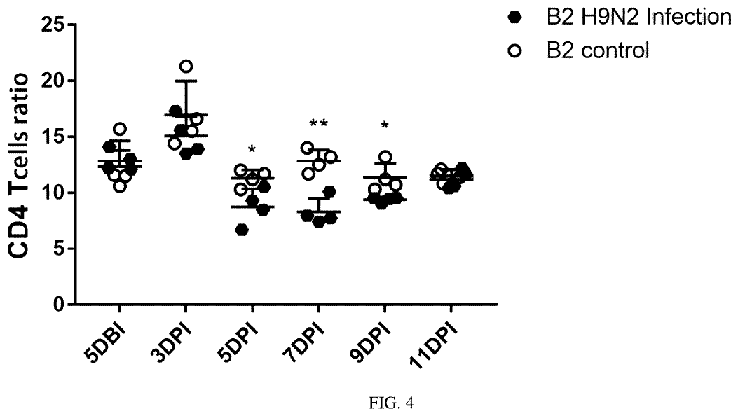

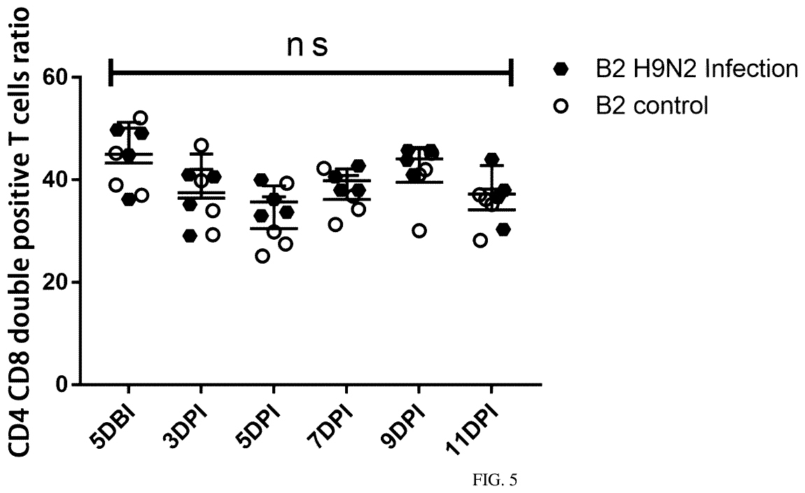

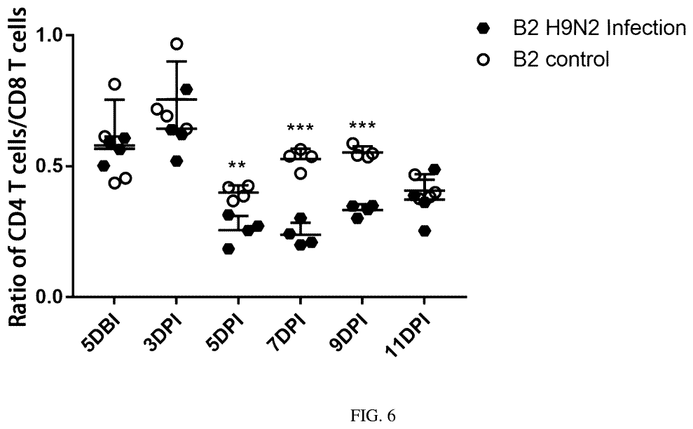

The changes of CD4⁺ T cell subtypes are illustrated in FIG. 4. After H9N2 AIV infection of B2 haplotype chickens, the proportion of CD4⁺ T cells decreases significantly (P<0.05) at 5 DPI, 7 DPI, 9 DPI and returns to a normal level at 11 DPI; the proportion changes of CD4⁺CD8α⁺ double positive T cells in PBMC from B2 haplotype chickens after challenge are shown in FIG. 5, with no statistical difference in the proportion of CD4⁺CD8α⁺ double positive T cells in the challenge group compared to that in the control group. Moreover, the changes of CD4⁺/CD8α⁺ T cells in B2 haplotype chicken PBMC after the challenge are shown in FIG. 6, and the CD4⁺/CD8α⁺ T cell ratios in the 5 DPI, 7 DPI and 9 DPI challenge groups are significantly lower than those in the control group, indicating that the organism is in an immunosuppressed state at this stage.

### 3.4 Expression of Immune-Related Genes in PBMC of B2 Haplotype Chickens after Infection

In order to further verify the influence of immune response in the process of H9N2 AIV infecting B2 haplotype chickens, the changes of mRNA expression of important immune genes in PBMC after 5 days of in vivo infection are detected by fluorescence quantitative PCR, which mainly includes three parts: natural immune-related genes, CTLs genes and Th2 genes.

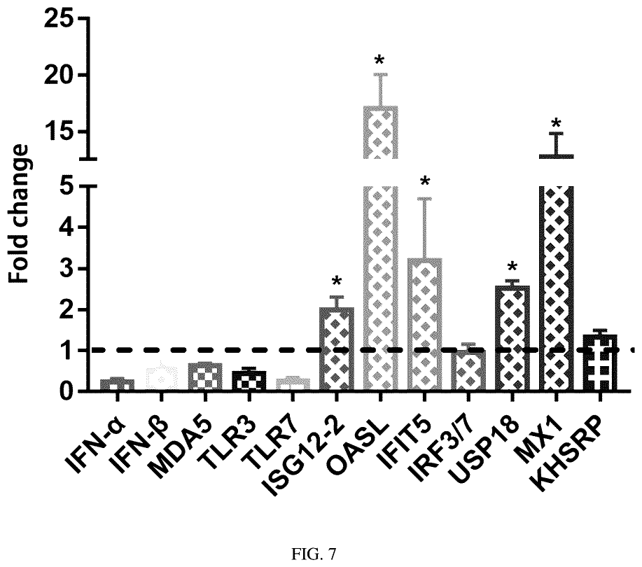

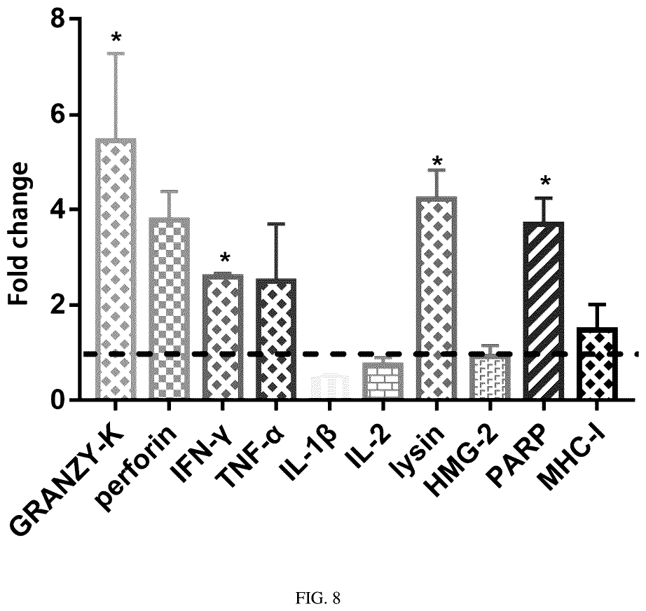

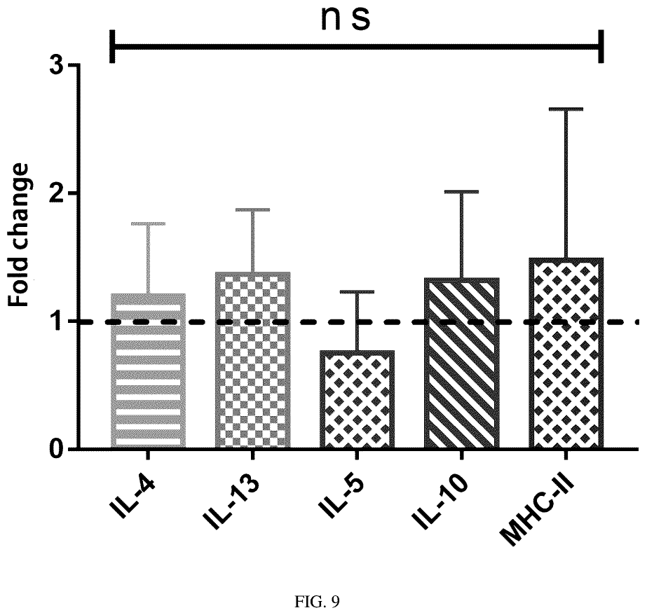

In the innate immunity gene fraction (FIG. 7), the expressions of the antiviral genes interferon-stimulated gene 12-2 (ISG12-2), 2′,5′ -oligoadenylate synthetase-like (OASL), interferon-induced proteins with tetratricopeptide repeats 5 (IFIT5), (ubiquitin specific peptidase 18) USP18 and myxovirus resistance 1 (MX1) significantly increased in the 5 DPI infected group compared to those in the control groups (P<0.05). As can be seen from the CTLs gene fraction (FIG. 8), the expressions of Granzyme k, IFN-γ, NK lysin, Poly-(ADP-ribose) Polymerase (PARP) increased significantly (P<0.05); and there no obvious changes of expression as detected in the Th2 gene (FIG. 9). Taken together with the increased proportion of CD8α⁺ T cells as shown in 3.3, it is further suggested that AIV infection has successfully activated the cytotoxic T cell immune response in B2 haplotype chickens. In summary, these results suggest that the model of H9N2 subtype AIV infection inducing cellular immune response in B2 haplotype SPF chickens (BW/G3) is successfully established.

### 3.5 Screening of Candidate Polypeptide Epitopes with Immunogenicity

According to the binding motif (A/V/I/L/P/S/G-X-X-X-X-X-X-V/I/L) of MHC class I molecular of B2 haplotype chicken, the candidate polypeptide epitopes for H9N2 subtype AIV are screened, and the screened peptides are shown in Table 5.

TABLE 5
Candidate Polypeptides with Immunogenicity
Screened According to the Motif

| Pool | | | | Pool | | | |
|---|---|---|---|---|---|---|---|
| serial | Peptide | Peptide | Sequence | serial | Peptide | Peptide | Sequence |
| number | name | sequence | ID | number | name | sequence | ID |
| Pool_1 | P1 | SAKEAQDVI | 75 | Pool_2 | P6 | LIGQGDVVL | 216 |
| | P2 | SAVLRGFLI | 76 | | P7 | ELVRKTRFL | 217 |
| | P3 | PIDNVMGMI | 77 | | P8 | VLTGNLQTL | 218 |
| | P4 | SLIIAARNI | 78 | | P9 | ILRKATKRL | 219 |
| | P5 | VLVNTYQWI | 79 | | P10 | AVKGIGTMV | 1 |
| Pool_3 | P11 | DVSFQGRGV | 2 | Pool_4 | P16 | MVGRRATAI | 80 |
| | P12 | GVRVSKMGV | 355 | | P17 | KVLFQNWGI | 81 |
| | P13 | RVRDQRGNV | 356 | | P18 | LVNTYQWII | 82 |
| | P14 | NVLLSPEEV | 357 | | P19 | NVRGSGMRI | 83 |
| | P15 | DVLGTFDTV | 358 | | P20 | YPITADKRI | 84 |
| Pool_5 | P21 | RLNPMHQLL | 220 | Pool_6 | P26 | IVVRGNSPV | 359 |
| | P22 | VLGKDAGAL | 221 | | P27 | NVLIGQGDV | 360 |
| | P23 | SVYIEVLHL | 222 | | P28 | GPVHFRNQV | 361 |
| | P24 | SVKREEEVL | 223 | | P29 | DPDEGTAGV | 362 |
| | P25 | FVNRANQRL | 224 | | P30 | TSTVHYPKV | 363 |
| Pool_7 | P31 | FPNEVGARI | 85 | Pool_8 | P36 | NVMGMIGIL | 225 |
| | P32 | MSQSRTREI | 86 | | P37 | VVVSIDRFL | 226 |
| | P33 | VSGKDEQSI | 87 | | P38 | AVLRGFLIL | 227 |
| | P34 | YSSSMMWEI | 88 | | P39 | APLMVAYML | 228 |
| | P35 | DSQTATKRI | 89 | | P40 | GPALSINEL | 229 |
| Pool_9 | P41 | LSAKEAQDV | 364 | Pool_10 | P46 | AGGTSSVYI | 90 |
| | P42 | SSVKREEEV | 365 | | P47 | IGGVRMVDI | 91 |
| | P43 | QSIAEAIIV | 366 | | P48 | GTFDTVQI | 92 |
| | P44 | YSSTERVVV | 367 | | P49 | KGEKANVLI | 93 |
| | P45 | VSIDRFLRV | 368 | | P50 | DAGSDRVMV | 94 |
| Pool_11 | P51 | TSSVYIEVL | 230 | Pool_12 | P56 | GGEVRNDDV | 369 |
| | P52 | VSADPLASL | 231 | | P57 | EGYEEFTMV | 370 |
| | P53 | QSRMQFSSL | 232 | | P58 | WGIEPIDNV | 371 |
| | P54 | ESAVLRGFL | 233 | | P59 | RGSGMRIVV | 372 |
| | P55 | LSINELSNL | 234 | | P60 | IGQGDVVLV | 373 |
| Pool_13 | P61 | VGRRATAIL | 95 | Pool_14 | P66 | GGVRMVDIL | 235 |
| | P62 | KATKRLIQL | 96 | | P67 | NLQTLKLRV | 236 |
| | P63 | KATKRLTVL | 97 | | P68 | VLFQQMRDV | 237 |
| | P64 | TAGVESAVL | 98 | | P69 | VLIGQGDVV | 238 |
| | P65 | DINPGHADL | 99 | | P70 | RVMVSPLAV | 239 |
| Pool_15 | P71 | VAYMLEREL | 374 | Pool_16 | P76 | RILTSESQL | 100 |
| | P72 | TGNLQTLKL | 375 | | P77 | DICKAAMGL | 101 |
| | P73 | LAKGEKANV | 376 | | P78 | MIKAVRGDL | 102 |
| | P74 | RATVSADPL | 377 | | P79 | EINGPESVL | 103 |
| | P75 | SADPLASLL | 378 | | P80 | LIIAARNIV | 104 |
| Pool_17 | P81 | PVAGGTSSV | 240 | Pool_18 | P86 | LLRTAVGQV | 379 |
| | P82 | AVRGDLNFV | 241 | | P87 | ASICTHLEV | 380 |
| | P83 | EAQDVIMEV | 242 | | P88 | NSICNTTGV | 381 |
| | P84 | CLLQSLQQI | 243 | | P89 | VSRPMFLYV | 382 |
| | P85 | SLQQIESMI | 244 | | P90 | ESRKLLLIV | 383 |
| Pool_19 | P91 | MAWTVVNSI | 105 | Pool_20 | P96 | DLEGLYEAI | 245 |
| | P92 | AAMDDFQLI | 106 | | P97 | GVTRREVHI | 246 |
| | P93 | KIKSEKTHI | 107 | | P98 | QVLSELQDI | 247 |
| | P94 | RIKTRLFTI | 108 | | P99 | DPSHEGEGI | 248 |
| | P95 | IIKPHKKGI | 109 | | P100 | EPRSLSCWI | 249 |
| Pool_21 | P101 | MATKADYTL | 384 | Pool_22 | P106 | EIGEDVAPI | 110 |
| | P102 | CAAMDDFQL | 385 | | P107 | ALLKHRFEI | 111 |
| | P103 | LAKSVFNSL | 386 | | P108 | LLKHRFEII | 112 |
| | P104 | SAESRKLLL | 387 | | P109 | EIGVTRREV | 113 |
| | P105 | NASWFNSFL | 388 | | P110 | PIGESPKGV | 114 |
| Pool_23 | P111 | KSEKTHIHI | 250 | Pool_24 | P116 | EITGTVRRL | 389 |
| | P112 | QSERGEETI | 251 | | P117 | FIIKGRSHL | 390 |
| | P113 | MSKEVNARI | 252 | | P118 | SIGKVCRTL | 391 |
| | P114 | SSWVELDEI | 253 | | P119 | LINDPWVLL | 392 |
| | P115 | DGFEPNGCI | 254 | | P120 | SLPPNFSSL | 393 |
| Pool_25 | P121 | SLENFRAYV | 115 | Pool_26 | P126 | EGIPLYDAI | 255 |
| | P122 | KLSQMSKEV | 116 | | P127 | FGWKEPNII | 256 |
| | P123 | ELDEIGEDV | 117 | | P128 | FLLMDALKL | 257 |
| | P124 | HLRNDTDVV | 118 | | P129 | PGTFDLEGL | 258 |
| | P125 | WGMEMRRCL | 119 | | P130 | ESSVKEKDL | 259 |
| Pool_27 | P131 | FLKTTPRPL | 394 | Pool_28 | P136 | IGKVCRTLL | 120 |
| | P132 | NSLYASPQL | 395 | | P137 | CVLEIGDML | 121 |
| | P133 | FSAESRKLL | 396 | | P138 | IVQALRDNL | 122 |
| | P134 | TGVEKPKFL | 397 | | P139 | ESGDPNALL | 123 |
| | P135 | KGINPNYLL | 398 | | P140 | CSORSKFLL | 124 |
| Pool_29 | P141 | VLEIGDMLL | 260 | Pool_30 | P146 | KGVYINTAL | 399 |
| | P142 | KLLLIVQAL | 261 | | P147 | YLLTWKQVL | 400 |
| | P143 | NLEPGTFDL | 262 | | P148 | SITIERMVL | 401 |
| | P144 | CLINDPWVL | 263 | | P149 | VGIDPFRLL | 402 |
| | P145 | GVYINTALL | 264 | | P150 | GIGTMVMEL | 403 |
| Pool_31 | P151 | TAGLTHLMI | 125 | Pool_32 | P156 | LSDNEGRLI | 265 |
| | P152 | AAGAAVKGI | 126 | | P157 | TSDMRTEII | 266 |
| | P153 | VAYERMCNI | 127 | | P158 | EGRLIONSI | 267 |
| | P154 | LILYDKEEI | 128 | | P159 | DGKWVRELI | 268 |
| | P155 | LIRMIKRGI | 129 | | P160 | IGTMVMELI | 269 |
| Pool_33 | P161 | LIFLARSAL | 404 | Pool_34 | P166 | RLIQNSITI | 130 |
| | P162 | KLSDNEGRL | 405 | | P167 | QLSTRGVQI | 131 |
| | P163 | HLMIWHSNL | 406 | | P168 | ELRSRYWAI | 132 |
| | P164 | FLARSALIL | 407 | | P169 | NLPFERATI | 133 |
| | P165 | CLPACVYGL | 408 | | P170 | SVGRMVSGI | 134 |
| Pool_35 | P171 | NATEIRASV | 270 | Pool_36 | P176 | LVGIDPFRL | 409 |
| | P172 | SALILRGSV | 271 | | P177 | CSLMQGSTL | 410 |
| | P173 | PACVYGLAV | 272 | | P178 | GSVAHKSCL | 411 |
| | P174 | SAAFEDLRV | 273 | | P179 | NGEDATAGL | 412 |
| | P175 | DPKKTGGPI | 274 | | P180 | PGNAEIEDL | 413 |
| Pool_37 | P181 | MVMELIRMI | 135 | Pool_38 | P186 | VSGIGRFYI | 275 |
| | P182 | RSGAAGAAV | 136 | | P187 | NPAHKSQLV | 276 |
| | P183 | ISVQPTFSV | 137 | | P188 | NSITIERMV | 277 |
| | P184 | RGQLSTRGV | 138 | | P189 | RASAGQISV | 278 |
| | P185 | DATAGLTHL | 139 | | P190 | IAIGSVSLI | 279 |
| Pool_39 | P191 | KADTRVLFI | 414 | Pool 40 | P196 | ASGKADTRV | 140 |
| | P192 | AIGSVSLII | 415 | | P197 | SSYVCSGLV | 141 |
| | P193 | AIICLLMQI | 416 | | P198 | RSGYETFRV | 142 |
| | P194 | SIGSWSKNI | 417 | | P199 | SGYETFRVV | 143 |
| | P195 | YINMADYSI | 418 | | P200 | SGYSGIFSV | 144 |
| Pool_41 | P201 | RVWWTSNSI | 280 | Pool_42 | P206 | HLGTKQVCI | 419 |
| | P202 | NPNQKIIAI | 281 | | P207 | VLFIREGKI | 420 |
| | P203 | APFSKDNSI | 282 | | P208 | SVSLIIAII | 421 |
| | P204 | GSVSLIIAI | 283 | | P209 | QVMPCEPII | 422 |
| | P205 | NSTIIEREI | 284 | | P210 | TVHLNSTII | 423 |
| Pool_43 | P211 | RGRPQEPRV | 145 | Pool_44 | P216 | GSNRPILYI | 285 |
| | P212 | FALGQGTTL | 146 | | P217 | KSQVNRQVI | 286 |
| | P213 | IIAIGSVSL | 147 | | P218 | FSVEGKKCI | 287 |
| | P214 | LIIAIICLL | 148 | | P219 | GSWPDGANI | 288 |
| | P215 | AILTTTMTL | 149 | | P220 | WAFDDGNDV | 289 |
| Pool_45 | P221 | ILERNTVHL | 424 | Pool_46 | P226 | SLIIAIICL | 150 |
| | P222 | IAIGSVSLI | 425 | | P227 | MATTTNPLI | 151 |
| | P223 | ASIIYDGML | 426 | | P228 | DLESLMEWI | 152 |
| | P224 | ESSYVCSGL | 427 | | P229 | ILSPLAKGI | 153 |
| | P225 | IGSWSKNIL | 428 | | P230 | ALASCMGLI | 154 |
| Pool_47 | P231 | GANINFMPV | 290 | Pool_48 | P236 | SGKADTRVL | 429 |
| | P232 | FSKDNSIRL | 291 | | P237 | PLAGSAQHV | 430 |
| | P233 | LVCATCEQI | 292 | | P238 | CSNPTNNQV | 431 |
| | P234 | PSGPLKAEI | 293 | | P239 | LSAGGDIWV | 432 |
| | P235 | LAKGILGFV | 294 | | P240 | PIILERNTV | 433 |
| Pool_49 | P241 | EVETYVLSI | 155 | Pool_50 | P246 | TAEGALGLV | 295 |
| | P242 | DPNNMDKAV | 156 | | P247 | VASQARQMV | 296 |
| | P243 | LGFVFTLTV | 157 | | P248 | LSIIPSGPL | 297 |
| | P244 | VIAANIIGI | 158 | | P249 | RGLORRRFV | 298 |
| | P245 | NIIGILHLI | 159 | | P250 | KAVKLYKKL | 299 |
| Pool_51 | P251 | CINGTCAVV | 434 | Pool_52 | P256 | GILHLILWI | 160 |
| | P252 | TLLMNELGV | 435 | | P257 | WIIRNWETV | 4 |
| | P253 | CLLMQIAIL | 436 | | P258 | PLVIAANII | 161 |
| | P254 | QAYQNRMGV | 437 | | P259 | DPLVIAANI | 162 |
| | P255 | EIAQRLEDV | 438 | | P260 | SGSSDPLVI | 163 |
| Pool_53 | P261 | GALASCMGL | 300 | Pool_54 | P266 | LIYNRMGTV | 439 |
| | P262 | GILGFVFTL | 301 | | P267 | LIRHENRMV | 440 |
| | P263 | ALSYSTGAL | 302 | | P268 | LLTEVETYV | 441 |
| | P264 | LSPLAKGIL | 303 | | P269 | SSTGLKDDL | 442 |
| | P265 | CSGSSDPLV | 304 | | P270 | LILWILDRL | 443 |
| Pool_55 | P271 | RIQIFPDTI | 164 | Pool_56 | P276 | IAANIIGIL | 305 |
| | P272 | RIKSNGNLI | 165 | | P277 | IIGILHLIL | 306 |
| | P273 | LIAPWYGHI | 166 | | P278 | YIVERPSAV | 307 |
| | P274 | KITSKVNNI | 167 | | P279 | GIKSLKLAV | 308 |
| | P275 | KIDDQIQDI | 168 | | P280 | AIDKITSKV | 309 |
| Pool_57 | P281 | ILHLIL WIL | 444 | Pool_58 | P286 | PLILDTCTI | 169 |
| | P282 | DGHFVNIEL | 445 | | P287 | RLNMINNKI | 170 |
| | P283 | TASLITILL | 446 | | P288 | LVNGLMGRI | 171 |
| | P284 | CATSLGHPL | 447 | | P289 | EVETRLNMI | 172 |
| | P285 | IAPWYGHIL | 448 | | P290 | VPSRSSRGI | 173 |
| Pool_59 | P291 | JIDHEFSEV | 310 | Pool_60 | P296 | IAMGFAAFL | 449 |
| | P292 | KILTIYSTV | 311 | | P297 | EINRTFKPL | 450 |
| | P293 | SLITILLVV | 312 | | P298 | RINYYWSVL | 451 |
| | P294 | TLTENNVPV | 313 | | P299 | YIGIKSLKL | 452 |
| | P295 | PLIGPRPLV | 314 | | P300 | FIEGGWSGL | 453 |
| Pool_61 | P301 | VSNADKICI | 174 | Pool_62 | P306 | DLKRGSCTV | 315 |
| | P302 | SSARSYQRI | 175 | | P307 | KLAVGLRNV | 316 |
| | P303 | LSGESHGRI | 176 | | P308 | TLDEHDANV | 317 |
| | P304 | SSRGIFGAI | 177 | | P309 | NVNNLYNKV | 318 |
| | P305 | ESKLERQKI | 178 | | P310 | ASLITILLV | 319 |
| Pool_63 | P311 | DIWAYNAEL | 454 | Pool_64 | P316 | KPLIGPRPL | 179 |
| | P312 | VLLENQKTL | 455 | | P317 | GPRPLVNGL | 180 |
| | P313 | PVTHAKELL | 456 | | P318 | NSTETVDTL | 181 |
| | P314 | SVLKPGQTL | 457 | | P319 | TSLGHPLIL | 182 |
| | P315 | VPVTHAKEL | 458 | | P320 | KSLKLAVGL | 183 |
| Pool_65 | P321 | QSTNSTETV | 320 | Pool_66 | P326 | GGREWSYIV | 459 |
| | P322 | YSTVASSLV | 321 | | P327 | NGLCYPGNV | 460 |
| | P323 | AATALANTI | 322 | | P328 | VPAEMLANI | 461 |
| | P324 | TANESGRLI | 323 | | P329 | LPSFGVSGI | 462 |
| | P325 | EAMVSRARI | 324 | | P330 | NPFVSHKEI | 463 |
| Pool_67 | P331 | ESEGTYKIL | 184 | Pool_68 | P336 | AIATPGMQI | 325 |
| | P332 | NGMLCATSL | 185 | | P337 | WIPKRNRSI | 326 |
| | P333 | YGNPSCDLL | 186 | | P338 | RIDFESGRI | 327 |
| | P334 | SGESHGRIL | 187 | | P339 | EIMKICSTI | 328 |
| | P335 | HGRILKTDL | 188 | | P340 | FLEESHPGI | 329 |
| Pool_69 | P341 | NSCLETMEI | 464 | Pool_70 | P346 | EGTYKILTI | 189 |
| | P342 | LSTVLGVSI | 465 | | P347 | WAYNAELLV | 190 |
| | P343 | QSSDDFALI | 466 | | P348 | GSCRCNICI | 191 |
| | P344 | SSYRRPVGI | 467 | | P349 | LGGREWSYI | 192 |
| | P345 | TGAPQLNPI | 468 | | P350 | TLLFLKVPV | 193 |
| Pool_71 | P351 | RLNKRSYLI | 330 | Pool_72 | P356 | RGKLKRRAI | 469 |
| | P352 | ILNTSQRGI | 331 | | P357 | DGGPNLYNI | 470 |
| | P353 | FVEALARSI | 332 | | P358 | TALANTIEV | 471 |
| | P354 | AVATTHSWI | 333 | | P359 | LIDFLKDVV | 472 |
| | P355 | NPRMFLAMI | 334 | | P360 | QIRGFVYFV | 473 |
| Pool_73 | P361 | RLIDFLKDV | 194 | Pool_74 | P366 | EIDSVNNAV | 335 |
| | P362 | KLEQSGLPV | 195 | | P367 | SSDDFALIV | 336 |
| | P363 | NPTLLFLKV | 196 | | P368 | ESADMSIGV | 337 |
| | P364 | RPVGISSMV | 197 | | P369 | VSHKEIDSV | 338 |
| | P365 | KSMKLRTQV | 198 | | P370 | PSSSYRRPV | 339 |
| Pool_75 | P371 | TIGKKKQRL | 474 | Pool_76 | P376 | QLNPIDGPL | 199 |
| | P372 | ISSMVEAMV | 475 | | P377 | LLIDGTASL | 200 |
| | P373 | MSRDWLMLI | 3 | | P378 | VLGVSILNL | 201 |
| | P374 | SGYAQTDCV | 476 | | P379 | NLHIPEVCL | 202 |
| | P375 | PGMQIRGFV | 477 | | P380 | DVNPTLLFL | 203 |
| Pool_77 | P381 | TATREGKHI | 340 | Pool_78 | P386 | LARSICEKL | 478 |
| | P382 | RAFTDEGAI | 341 | | P387 | VIFNRLEAL | 479 |
| | P383 | TLKANFSVI | 342 | | P388 | AIVGEISPL | 480 |
| | P384 | RSSTLGLDI | 343 | | P389 | RLRRDQKSL | 481 |
| | P385 | SIRLMDRCL | 344 | | P390 | SLRGRSSTL | 482 |
| Pool_79 | P391 | CVLEAMAFL | 204 | Pool_80 | P396 | CIRMDQAIV | 345 |
| | P392 | FVANFSMEL | 205 | | P397 | QVDCFLWHV | 346 |
| | P393 | LVSDGGPNL | 206 | | P398 | LPGHTDKDV | 347 |
| | P394 | QPEWFRNVL | 207 | | P399 | AIVDKNITL | 348 |
| | P395 | GPATAQMAL | 208 | | P400 | EGKHIVERI | 349 |
| Pool_81 | P401 | DVKNAIEVL | 483 | Pool_82 | P406 | IPKRNRSIL | 209 |
| | P402 | SSFQVDCFL | 484 | | P407 | ESGRLIDFL | 210 |
| | P403 | ASVPAPRYL | 485 | | P408 | KICSTIEEL | 211 |
| | P404 | RGRSSTLGL | 486 | | P409 | KIEKIRPLL | 212 |
| | P405 | QLSOKFEEI | 487 | | P410 | QLRSSSEDL | 213 |
| Pool_83 | P411 | SSEDLNGMI | 350 | Pool_84 | P416 | LLLEVEQEI | 488 |
| | P412 | QALQLLLEV | 351 | | P417 | TVSSFQDIL | 489 |
| | P413 | EIR WLIEEV | 352 | | P418 | EAAMRMGDL | 490 |
| | P414 | KLFSKQEWI | 353 | | P419 | LIEEVRHRL | 491 |
| | P415 | RVGMHKRIV | 354 | | P420 | EIRTFSFQL | 492 |
| Pool_85 | P421 | SLKLYRDSL | 214 | | | | |
| | P422 | RIVYWKQWL | 215 | | | | |

### 3.6 Immunogenicity Detection of Candidate Peptides

### 3.6.1 ELISpot assay for Immunogenicity Detection of Peptide Pools

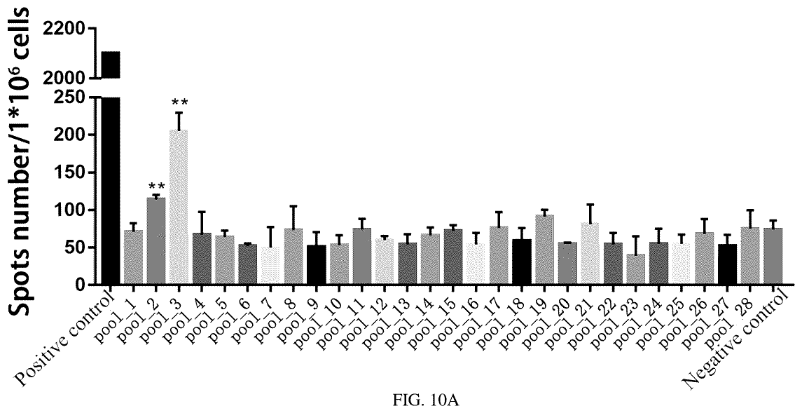

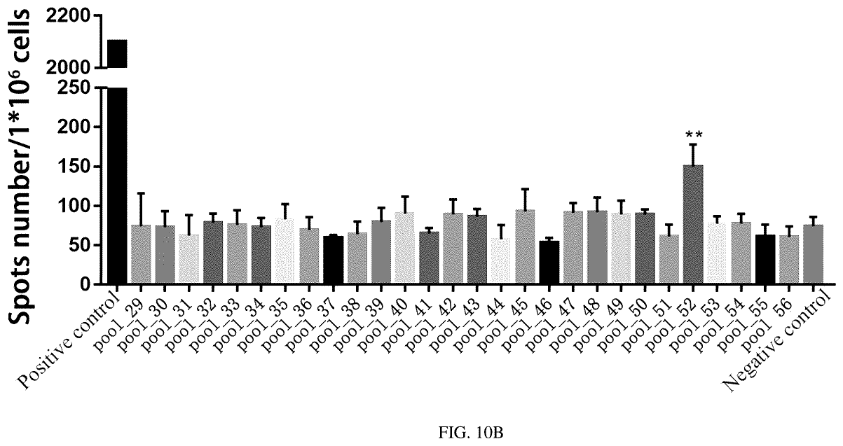

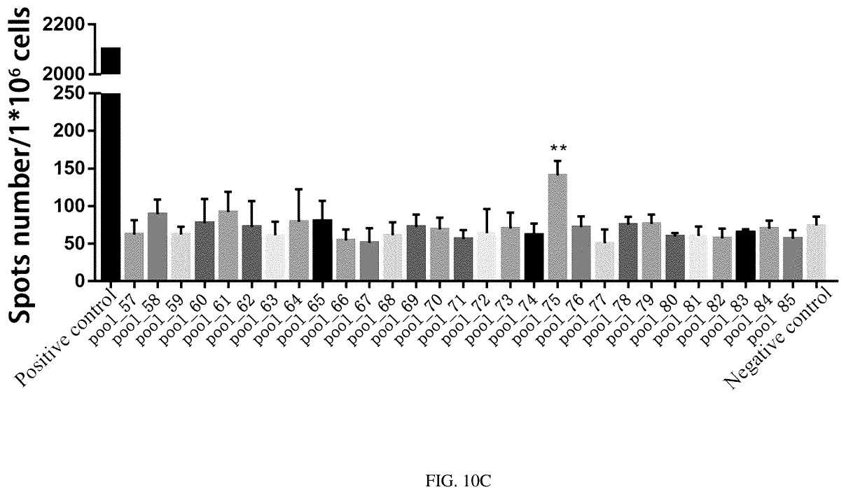

The synthesized peptides are mixed according to the requirement of five peptides as a pool, and the spleen lymphocytes of B2 haplotype chickens infected with H9N2 AIV for 28 days are stimulated respectively. As shown in FIGS. 10A-10C, it is found by statistical analysis that pool_2, pool_3, pool_52 and pool_75 significantly stimulate the production of IFN-γ spots by splenic lymphocytes, indicating the presence of immunogenic epitopes in the peptides that constitutes the above-mentioned peptide pools.

### 3.6.2 ELISpot Assay for Immunogenicity Detection of Polypeptide

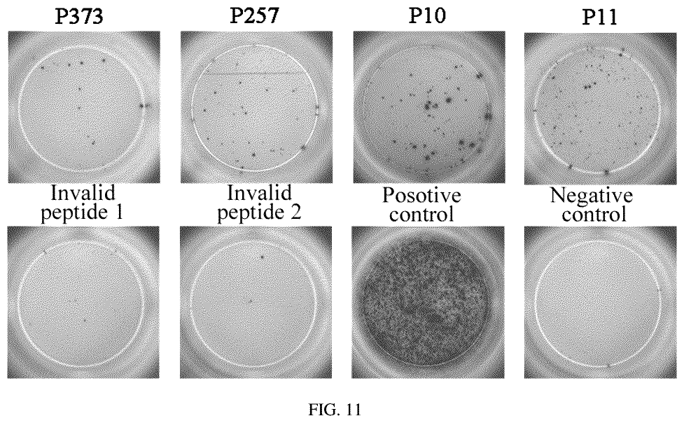

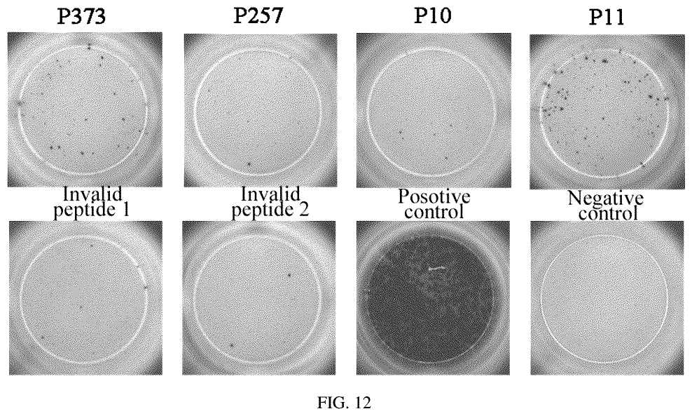

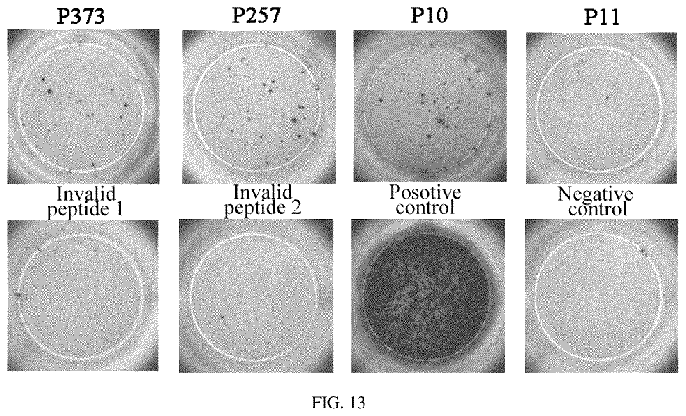

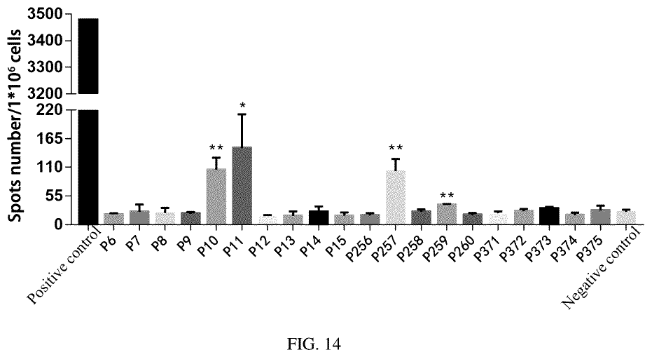

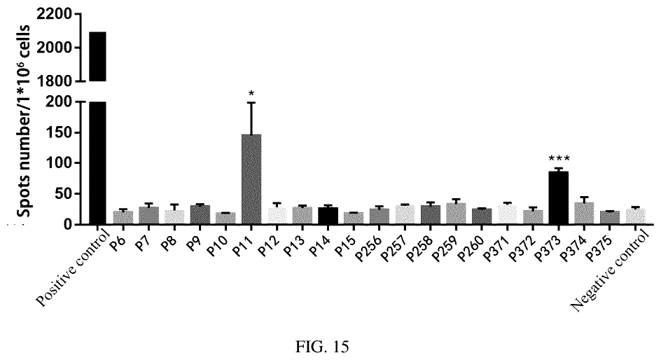

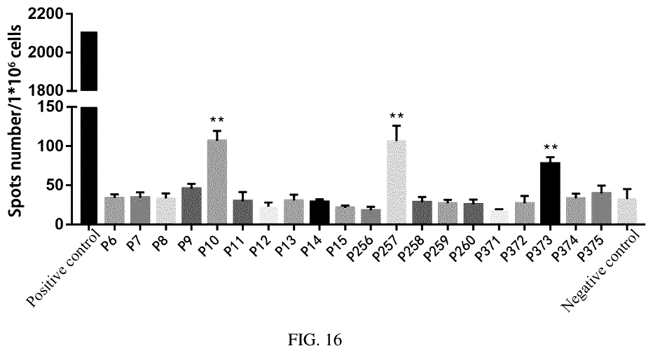

FIGS. 11-13 suggest that a single peptide that is immunogenic may also stimulate IFN-γ production by splenic lymphocytes; according to the criteria provided in the reference (Identification of novel avian influenza virus derived CD8⁺ T-cell epitopes), a peptide that causes significant IFN-γ production in at least 2 of 3 chickens is considered immunogenic compared to a negative control, and four peptides, P10, P11, P373 and P257, satisfy this criteria and may be considered as B2 haplotype restrictive H9N2 AIV T-cell epitopes, as shown in FIGS. 14-16 (Shown in Table 6).

TABLE 6
Epitope Information for Four H9N2 Subtype AIV
T Cells Targeting Haplotype B2 Chickens

| | | Protein in | |
|---|---|---|---|
| | | which the | |
| Polypeptide | Polypeptide | polypeptide | Protein |
| name | sequence | is located | sites |
| P10 | AVKGIGTMV | NP | 182-190 |
| | (SEQ ID NO. 1) | | |
| P11 | DVSFQGRGV | NP | 455-463 |
| | (SEQ ID NO. 2) | | |
| P373 | MSRDWLMLI | NS1 | 98-106 |
| | (SEQ ID NO. 3) | | |
| P257 | WIIRNWETV | PB2 | 552-560 |
| | (SEQ ID NO. 4) | | |

The above-mentioned embodiments only describe the preferred mode of the present application, and do not limit the scope of the present application. Under the premise of not departing from the design spirit of the present application, various modifications and improvements made by ordinary technicians in the field to the technical scheme of the present application shall fall within the protection scope determined by the claims of the present application.

What is claimed is:

1. A restrictive epitope peptide of a major histocompatibility complex (MHC) B2 of an H9N2 subtype Avian influenza virus (AIV), wherein the restrictive epitope peptide has an amino acid sequence as shown in SEQ ID NO.1, SEQ ID NO.2, SEQ ID NO.3 or SEQ ID No.4.

2. An application of the restrictive epitope peptide according to claim 1 in preparing a vaccine against H9N2 subtype AIV.

3. A vaccine against H9N2 subtype AIV, comprising the restrictive epitope peptide according to claim 1.


SEQUENCE LISTING (492 sequences)

SEQ ID NO: 1 - protein, 9 aa, synthetic construct
AVKGIGTMV

SEQ ID NO: 2 - protein, 9 aa, synthetic construct
DVSFQGRGV

SEQ ID NO: 3 - protein, 9 aa, synthetic construct
MSRDWLMLI

SEQ ID NO: 4 - protein, 9 aa, synthetic construct
WIIRNWETV

SEQ ID NO: 5 - DNA, 20 bp, synthetic construct
  source 1..20: mol_type = other DNA
gaacatcatcccagcgtcca

SEQ ID NO: 6 - DNA, 20 bp, synthetic construct
  source 1..20: mol_type = other DNA
cggcaggtcaggtcaacaac

SEQ ID NO: 7 - DNA, 20 bp, synthetic construct
  source 1..20: mol_type = other DNA
cgggaagcaactgttgaaat

SEQ ID NO: 8 - DNA, 20 bp, synthetic construct
  source 1..20: mol_type = other DNA
gagtctcccttgcaagcatc

SEQ ID NO: 9 - DNA, 19 bp, synthetic construct
  source 1..19: mol_type = other DNA
atggcgcaggtgacagtga

SEQ ID NO: 10 - DNA, 19 bp, synthetic construct
  source 1..19: mol_type = other DNA
tggcctgcaccggtaattc

SEQ ID NO: 11 - DNA, 22 bp, synthetic construct
  source 1..22: mol_type = other DNA
cctccaacacctcttcaacatg

SEQ ID NO: 12 - DNA, 15 bp, synthetic construct
  source 1..15: mol_type = other DNA
tggcgtgcggtcaat

SEQ ID NO: 13 - DNA, 22 bp, synthetic construct
  source 1..22: mol_type = other DNA
gctgttctatgaccgcccagtt

SEQ ID NO: 14 - DNA, 21 bp, synthetic construct
  source 1..21: mol_type = other DNA
aacaaccagctatgcacccca

SEQ ID NO: 15 - DNA, 20 bp, synthetic construct
  source 1..20: mol_type = other DNA
ggtcaacatcgccacctaca

SEQ ID NO: 16 - DNA, 22 bp, synthetic construct
  source 1..22: mol_type = other DNA
catacgagatggaaaccagcaa

SEQ ID NO: 17 - DNA, 25 bp, synthetic construct
  source 1..25: mol_type = other DNA
gctaatgactacagcttatggagca

SEQ ID NO: 18 - DNA, 23 bp, synthetic construct
  source 1..23: mol_type = other DNA
tgggtctcagttggtgtgtagag

SEQ ID NO: 19 - DNA, 23 bp, synthetic construct
  source 1..23: mol_type = other DNA
gatggttcagctgcgtgggatgc

SEQ ID NO: 20 - DNA, 20 bp, synthetic construct
  source 1..20: mol_type = other DNA
ctgccggagcttcttcaaca

SEQ ID NO: 21 - DNA, 19 bp, synthetic construct
  source 1..19: mol_type = other DNA
agagcacaagaagaagcac

SEQ ID NO: 22 - DNA, 22 bp, synthetic construct
  source 1..22: mol_type = other DNA
gtcttttaggagcgttggggtc

SEQ ID NO: 23 - DNA, 20 bp, synthetic construct
  source 1..20: mol_type = other DNA
aagaaggggaagggctacaa

SEQ ID NO: 24 - DNA, 20 bp, synthetic construct
  source 1..20: mol_type = other DNA
aagcagtgcaggcaaagaat

SEQ ID NO: 25 - DNA, 18 bp, synthetic construct
  source 1..18: mol_type = other DNA
gacagccaacgccaaagc

SEQ ID NO: 26 - DNA, 20 bp, synthetic construct
  source 1..20: mol_type = other DNA
gtcgctgctgtccaagcatt

SEQ ID NO: 27 - DNA, 22 bp, synthetic construct
  source 1..22: mol_type = other DNA
gcccacacactccaaaacactg

SEQ ID NO: 28 - DNA, 21 bp, synthetic construct
  source 1..21: mol_type = other DNA
ttgatgctgaggtgagcgttg

SEQ ID NO: 29 - DNA, 20 bp, synthetic construct
  source 1..20: mol_type = other DNA
aaatccctcctcgccaatct

SEQ ID NO: 30 - DNA, 22 bp, synthetic construct
  source 1..22: mol_type = other DNA
ccctcacggtcttctccataaa

SEQ ID NO: 31 - DNA, 20 bp, synthetic construct
  source 1..20: mol_type = other DNA
agcagatcaaggagacgttc

SEQ ID NO: 32 - DNA, 20 bp, synthetic construct
  source 1..20: mol_type = other DNA
atcagcaggtactcctcgat

SEQ ID NO: 33 - DNA, 23 bp, synthetic construct
  source 1..23: mol_type = other DNA
acaatggcagattgtagtcacct

SEQ ID NO: 34 - DNA, 23 bp, synthetic construct
  source 1..23: mol_type = other DNA
gcacaatcctggtttcagtttag

SEQ ID NO: 35 - DNA, 20 bp, synthetic construct
  source 1..20: mol_type = other DNA
tctggacttctctaacaaca

SEQ ID NO: 36 - DNA, 23 bp, synthetic construct
  source 1..23: mol_type = other DNA
aatctcattctcattcatcatca

SEQ ID NO: 37 - DNA, 20 bp, synthetic construct
  source 1..20: mol_type = other DNA
aagaaggggaagggctacaa

SEQ ID NO: 38 - DNA, 20 bp, synthetic construct
  source 1..20: mol_type = other DNA
aagcagtgcaggcaaagaat

SEQ ID NO: 39 - DNA, 20 bp, synthetic construct
  source 1..20: mol_type = other DNA
ctcgaggtcatgatcagcaa

SEQ ID NO: 40 - DNA, 20 bp, synthetic construct
  source 1..20: mol_type = other DNA
tgtaaacgtctcccctttgg

SEQ ID NO: 41 - DNA, 17 bp, synthetic construct
  source 1..17: mol_type = other DNA
tcgaggagtgacgggtg

SEQ ID NO: 42 - DNA, 21 bp, synthetic construct
  source 1..21: mol_type = other DNA
actatccggatgctctccatc

SEQ ID NO: 43 - DNA, 23 bp, synthetic construct
  source 1..23: mol_type = other DNA
ggaacggcactgttgaaaaataa

SEQ ID NO: 44 - DNA, 23 bp, synthetic construct
  source 1..23: mol_type = other DNA
ttctccctctcctgtcagttgtg

SEQ ID NO: 45 - DNA, 20 bp, synthetic construct
  source 1..20: mol_type = other DNA
ctgcccttgctctcctctgt

SEQ ID NO: 46 - DNA, 22 bp, synthetic construct
  source 1..22: mol_type = other DNA
cctgcactcctctgttgagctt

SEQ ID NO: 47 - DNA, 20 bp, synthetic construct
  source 1..20: mol_type = other DNA
actcatgtcgaggggattca

SEQ ID NO: 48 - DNA, 20 bp, synthetic construct
  source 1..20: mol_type = other DNA
tgtagacaccaggaccacca

SEQ ID NO: 49 - DNA, 21 bp, synthetic construct
  source 1..21: mol_type = other DNA
ggacgaccacgatctctgtgt

SEQ ID NO: 50 - DNA, 24 bp, synthetic construct
  source 1..24: mol_type = other DNA
cacctgtctggtctgcatgttatc

SEQ ID NO: 51 - DNA, 16 bp, synthetic construct
  source 1..16: mol_type = other DNA
aactccgatgccagtg

SEQ ID NO: 52 - DNA, 16 bp, synthetic construct
  source 1..16: mol_type = other DNA
ttggtgtctgccttgt

SEQ ID NO: 53 - DNA, 20 bp, synthetic construct
  source 1..20: mol_type = other DNA
catcatgaagcattccatct

SEQ ID NO: 54 - DNA, 20 bp, synthetic construct
  source 1..20: mol_type = other DNA
cttccaagggatcttcattt

SEQ ID NO: 55 - DNA, 20 bp, synthetic construct
  source 1..20: mol_type = other DNA
tctttacattgacttccgac

SEQ ID NO: 56 - DNA, 20 bp, synthetic construct
  source 1..20: mol_type = other DNA
tcctcccaacatagtacaag

SEQ ID NO: 57 - DNA, 20 bp, synthetic construct
  source 1..20: mol_type = other DNA
aagcctgagcatgagcagaa

SEQ ID NO: 58 - DNA, 23 bp, synthetic construct
  source 1..23: mol_type = other DNA
tctcaggctgtcaacaagatcaa

SEQ ID NO: 59 - DNA, 22 bp, synthetic construct
  source 1..22: mol_type = other DNA
agatgttgaagccgaagtaccc

SEQ ID NO: 60 - DNA, 20 bp, synthetic construct
  source 1..20: mol_type = other DNA
ctgaagtcctccctgcctgt

SEQ ID NO: 61 - DNA, 19 bp, synthetic construct
  source 1..19: mol_type = other DNA
tcaatgggtggcaaaggag

SEQ ID NO: 62 - DNA, 25 bp, synthetic construct
  source 1..25: mol_type = other DNA
tacagggagagcaaagaagagaaga

SEQ ID NO: 63 - DNA, 22 bp, synthetic construct
  source 1..22: mol_type = other DNA
cagaatttaatgccggctatgc

SEQ ID NO: 64 - DNA, 26 bp, synthetic construct
  source 1..26: mol_type = other DNA
tgcaagtaaagccaaaagataagtgt

SEQ ID NO: 65 - DNA, 20 bp, synthetic construct
  source 1..20: mol_type = other DNA
caacgtgggaagaggagaaa

SEQ ID NO: 66 - DNA, 20 bp, synthetic construct
  source 1..20: mol_type = other DNA
acttcatgagcggagaagga

SEQ ID NO: 67 - DNA, 21 bp, synthetic construct
  source 1..21: mol_type = other DNA
actgaccagcccaggaactct

SEQ ID NO: 68 - DNA, 20 bp, synthetic construct
  source 1..20: mol_type = other DNA
aaggctttcccaaccacaaa

SEQ ID NO: 69 - DNA, 21 bp, synthetic construct
  source 1..21: mol_type = other DNA
ggtccacggttatggtgaaag

SEQ ID NO: 70 - DNA, 23 bp, synthetic construct
  source 1..23: mol_type = other DNA
ggtcagaaatcacaactcaagca

SEQ ID NO: 71 - DNA, 22 bp, synthetic construct
  source 1..22: mol_type = other DNA
cagcggggaaatgattaagaag

SEQ ID NO: 72 - DNA, 20 bp, synthetic construct
  source 1..20: mol_type = other DNA
tttgtgtgtggggatggaga

SEQ ID NO: 73 - DNA, 22 bp, synthetic construct
  source 1..22: mol_type = other DNA
attgtggaggagctgggaggaa

SEQ ID NO: 74 - DNA, 21 bp, synthetic construct
  source 1..21: mol_type = other DNA
aggcttgctgcacttcccatc

SEQ ID NO: 75 - protein, 9 aa, synthetic construct
SAKEAQDVI

SEQ ID NO: 76 - protein, 9 aa, synthetic construct
SAVLRGFLI

SEQ ID NO: 77 - protein, 9 aa, synthetic construct
PIDNVMGMI

SEQ ID NO: 78 - protein, 9 aa, synthetic construct
SLIIAARNI

SEQ ID NO: 79 - protein, 9 aa, synthetic construct
VLVNTYQWI

SEQ ID NO: 80 - protein, 9 aa, synthetic construct
MVGRRATAI

SEQ ID NO: 81 - protein, 9 aa, synthetic construct
KVLFQNWGI

SEQ ID NO: 82 - protein, 9 aa, synthetic construct
LVNTYQWII

SEQ ID NO: 83 - protein, 9 aa, synthetic construct
NVRGSGMRI

SEQ ID NO: 84 - protein, 9 aa, synthetic construct
YPITADKRI

SEQ ID NO: 85 - protein, 9 aa, synthetic construct
FPNEVGARI

SEQ ID NO: 86 - protein, 9 aa, synthetic construct
MSQSRTREI

SEQ ID NO: 87 - protein, 9 aa, synthetic construct
VSGKDEQSI

SEQ ID NO: 88 - protein, 9 aa, synthetic construct
YSSSMMWEI

SEQ ID NO: 89 - protein, 9 aa, synthetic construct
DSQTATKRI

SEQ ID NO: 90 - protein, 9 aa, synthetic construct
AGGTSSVYI

SEQ ID NO: 91 - protein, 9 aa, synthetic construct
IGGVRMVDI

SEQ ID NO: 92 - protein, 8 aa, synthetic construct
GTFDTVQI

SEQ ID NO: 93 - protein, 9 aa, synthetic construct
KGEKANVLI

SEQ ID NO: 94 - protein, 9 aa, synthetic construct
DAGSDRVMV

SEQ ID NO: 95 - protein, 9 aa, synthetic construct
VGRRATAIL

SEQ ID NO: 96 - protein, 9 aa, synthetic construct
KATKRLIQL

SEQ ID NO: 97 - protein, 9 aa, synthetic construct
KATKRLTVL

SEQ ID NO: 98 - protein, 9 aa, synthetic construct
TAGVESAVL

SEQ ID NO: 99 - protein, 9 aa, synthetic construct
DINPGHADL

SEQ ID NO: 100 - protein, 9 aa, synthetic construct
RILTSESQL

SEQ ID NO: 101 - protein, 9 aa, synthetic construct
DICKAAMGL

SEQ ID NO: 102 - protein, 9 aa, synthetic construct
MIKAVRGDL

SEQ ID NO: 103 - protein, 9 aa, synthetic construct
EINGPESVL

SEQ ID NO: 104 - protein, 9 aa, synthetic construct
LIIAARNIV

SEQ ID NO: 105 - protein, 9 aa, synthetic construct
MAWTVVNSI

SEQ ID NO: 106 - protein, 9 aa, synthetic construct
AAMDDFQLI

SEQ ID NO: 107 - protein, 9 aa, synthetic construct
KIKSEKTHI

SEQ ID NO: 108 - protein, 9 aa, synthetic construct
RIKTRLFTI

SEQ ID NO: 109 - protein, 9 aa, synthetic construct
IIKPHKKGI

SEQ ID NO: 110 - protein, 9 aa, synthetic construct
EIGEDVAPI

SEQ ID NO: 111 - protein, 9 aa, synthetic construct
ALLKHRFEI

SEQ ID NO: 112 - protein, 9 aa, synthetic construct
LLKHRFEII

SEQ ID NO: 113 - protein, 9 aa, synthetic construct
EIGVTRREV

SEQ ID NO: 114 - protein, 9 aa, synthetic construct
PIGESPKGV

SEQ ID NO: 115 - protein, 9 aa, synthetic construct
SLENFRAYV

SEQ ID NO: 116 - protein, 9 aa, synthetic construct
KLSQMSKEV

SEQ ID NO: 117 - protein, 9 aa, synthetic construct
ELDEIGEDV

SEQ ID NO: 118 - protein, 9 aa, synthetic construct
HLRNDTDVV

SEQ ID NO: 119 - protein, 9 aa, synthetic construct
WGMEMRRCL

SEQ ID NO: 120 - protein, 9 aa, synthetic construct
IGKVCRTLL

SEQ ID NO: 121 - protein, 9 aa, synthetic construct
CVLEIGDML

SEQ ID NO: 122 - protein, 9 aa, synthetic construct
IVQALRDNL

SEQ ID NO: 123 - protein, 9 aa, synthetic construct
ESGDPNALL

SEQ ID NO: 124 - protein, 9 aa, synthetic construct
CSQRSKFLL

SEQ ID NO: 125 - protein, 9 aa, synthetic construct
TAGLTHLMI

SEQ ID NO: 126 - protein, 9 aa, synthetic construct
AAGAAVKGI

SEQ ID NO: 127 - protein, 9 aa, synthetic construct
VAYERMCNI

SEQ ID NO: 128 - protein, 9 aa, synthetic construct
LILYDKEEI

SEQ ID NO: 129 - protein, 9 aa, synthetic construct
LIRMIKRGI

SEQ ID NO: 130 - protein, 9 aa, synthetic construct
RLIQNSITI

SEQ ID NO: 131 - protein, 9 aa, synthetic construct
QLSTRGVQI

SEQ ID NO: 132 - protein, 9 aa, synthetic construct
ELRSRYWAI

SEQ ID NO: 133 - protein, 9 aa, synthetic construct
NLPFERATI

SEQ ID NO: 134 - protein, 9 aa, synthetic construct
SVGRMVSGI

SEQ ID NO: 135 - protein, 9 aa, synthetic construct
MVMELIRMI

SEQ ID NO: 136 - protein, 9 aa, synthetic construct
RSGAAGAAV

SEQ ID NO: 137 - protein, 9 aa, synthetic construct
ISVQPTFSV

SEQ ID NO: 138 - protein, 9 aa, synthetic construct
RGQLSTRGV

SEQ ID NO: 139 - protein, 9 aa, synthetic construct
DATAGLTHL

SEQ ID NO: 140 - protein, 9 aa, synthetic construct
ASGKADTRV

SEQ ID NO: 141 - protein, 9 aa, synthetic construct
SSYVCSGLV

SEQ ID NO: 142 - protein, 9 aa, synthetic construct
RSGYETFRV

SEQ ID NO: 143 - protein, 9 aa, synthetic construct
SGYETFRVV

SEQ ID NO: 144 - protein, 9 aa, synthetic construct
SGYSGIFSV

SEQ ID NO: 145 - protein, 9 aa, synthetic construct
RGRPQEPRV

SEQ ID NO: 146 - protein, 9 aa, synthetic construct
FALGQGTTL

SEQ ID NO: 147 - protein, 9 aa, synthetic construct
IIAIGSVSL

SEQ ID NO: 148 - protein, 9 aa, synthetic construct
LIIAIICLL

SEQ ID NO: 149 - protein, 9 aa, synthetic construct
AILTTTMTL

SEQ ID NO: 150 - protein, 9 aa, synthetic construct
SLIIAIICL

SEQ ID NO: 151 - protein, 9 aa, synthetic construct
MATTTNPLI

SEQ ID NO: 152 - protein, 9 aa, synthetic construct
DLESLMEWI

SEQ ID NO: 153 - protein, 9 aa, synthetic construct
ILSPLAKGI

SEQ ID NO: 154 - protein, 9 aa, synthetic construct
ALASCMGLI

SEQ ID NO: 155 - protein, 9 aa, synthetic construct
EVETYVLSI

SEQ ID NO: 156 - protein, 9 aa, synthetic construct
DPNNMDKAV

SEQ ID NO: 157 - protein, 9 aa, synthetic construct
LGFVFTLTV

SEQ ID NO: 158 - protein, 9 aa, synthetic construct
VIAANIIGI

SEQ ID NO: 159 - protein, 9 aa, synthetic construct
NIIGILHLI

SEQ ID NO: 160 - protein, 9 aa, synthetic construct
GILHLILWI

SEQ ID NO: 161 - protein, 9 aa, synthetic construct
PLVIAANII

SEQ ID NO: 162 - protein, 9 aa, synthetic construct
DPLVIAANI

SEQ ID NO: 163 - protein, 9 aa, synthetic construct
SGSSDPLVI

SEQ ID NO: 164 - protein, 9 aa, synthetic construct
RIQIFPDTI

SEQ ID NO: 165 - protein, 9 aa, synthetic construct
RIKSNGNLI

SEQ ID NO: 166 - protein, 9 aa, synthetic construct
LIAPWYGHI

SEQ ID NO: 167 - protein, 9 aa, synthetic construct
KITSKVNNI

SEQ ID NO: 168 - protein, 9 aa, synthetic construct
KIDDQIQDI

SEQ ID NO: 169 - protein, 9 aa, synthetic construct
PLILDTCTI

SEQ ID NO: 170 - protein, 9 aa, synthetic construct
RLNMINNKI

SEQ ID NO: 171 - protein, 9 aa, synthetic construct
LVNGLMGRI

SEQ ID NO: 172 - protein, 9 aa, synthetic construct
EVETRLNMI

SEQ ID NO: 173 - protein, 9 aa, synthetic construct
VPSRSSRGI

SEQ ID NO: 174 - protein, 9 aa, synthetic construct
VSNADKICI

SEQ ID NO: 175 - protein, 9 aa, synthetic construct
SSARSYQRI

SEQ ID NO: 176 - protein, 9 aa, synthetic construct
LSGESHGRI

SEQ ID NO: 177 - protein, 9 aa, synthetic construct
SSRGIFGAI

SEQ ID NO: 178 - protein, 9 aa, synthetic construct
ESKLERQKI

SEQ ID NO: 179 - protein, 9 aa, synthetic construct
KPLIGPRPL

SEQ ID NO: 180 - protein, 9 aa, synthetic construct
GPRPLVNGL

SEQ ID NO: 181 - protein, 9 aa, synthetic construct
NSTETVDTL

SEQ ID NO: 182 - protein, 9 aa, synthetic construct
TSLGHPLIL

SEQ ID NO: 183 - protein, 9 aa, synthetic construct
KSLKLAVGL

SEQ ID NO: 184 - protein, 9 aa, synthetic construct
ESEGTYKIL

SEQ ID NO: 185 - protein, 9 aa, synthetic construct
NGMLCATSL

SEQ ID NO: 186 - protein, 9 aa, synthetic construct
YGNPSCDLL

SEQ ID NO: 187 - protein, 9 aa, synthetic construct
SGESHGRIL

SEQ ID NO: 188 - protein, 9 aa, synthetic construct
HGRILKTDL

SEQ ID NO: 189 - protein, 9 aa, synthetic construct
EGTYKILTI

SEQ ID NO: 190 - protein, 9 aa, synthetic construct
WAYNAELLV

SEQ ID NO: 191 - protein, 9 aa, synthetic construct
GSCRCNICI

SEQ ID NO: 192 - protein, 9 aa, synthetic construct
LGGREWSYI

SEQ ID NO: 193 - protein, 9 aa, synthetic construct
TLLFLKVPV

SEQ ID NO: 194 - protein, 9 aa, synthetic construct
RLIDFLKDV

SEQ ID NO: 195 - protein, 9 aa, synthetic construct
KLEQSGLPV

SEQ ID NO: 196 - protein, 9 aa, synthetic construct
NPTLLFLKV

SEQ ID NO: 197 - protein, 9 aa, synthetic construct
RPVGISSMV

SEQ ID NO: 198 - protein, 9 aa, synthetic construct
KSMKLRTQV

SEQ ID NO: 199 - protein, 9 aa, synthetic construct
QLNPIDGPL

SEQ ID NO: 200 - protein, 9 aa, synthetic construct
LLIDGTASL

SEQ ID NO: 201 - protein, 9 aa, synthetic construct
VLGVSILNL

SEQ ID NO: 202 - protein, 9 aa, synthetic construct
NLHIPEVCL

SEQ ID NO: 203 - protein, 9 aa, synthetic construct
DVNPTLLFL

SEQ ID NO: 204 - protein, 9 aa, synthetic construct
CVLEAMAFL

SEQ ID NO: 205 - protein, 9 aa, synthetic construct
FVANFSMEL

SEQ ID NO: 206 - protein, 9 aa, synthetic construct
LVSDGGPNL

SEQ ID NO: 207 - protein, 9 aa, synthetic construct
QPEWFRNVL

SEQ ID NO: 208 - protein, 9 aa, synthetic construct
GPATAQMAL

SEQ ID NO: 209 - protein, 9 aa, synthetic construct
IPKRNRSIL

SEQ ID NO: 210 - protein, 9 aa, synthetic construct
ESGRLIDFL

SEQ ID NO: 211 - protein, 9 aa, synthetic construct
KICSTIEEL

SEQ ID NO: 212 - protein, 9 aa, synthetic construct
KIEKIRPLL

SEQ ID NO: 213 - protein, 9 aa, synthetic construct
QLRSSSEDL

SEQ ID NO: 214 - protein, 9 aa, synthetic construct
SLKLYRDSL

SEQ ID NO: 215 - protein, 9 aa, synthetic construct
RIVYWKQWL

SEQ ID NO: 216 - protein, 9 aa, synthetic construct
LIGQGDVVL

SEQ ID NO: 217 - protein, 9 aa, synthetic construct
ELVRKTRFL

SEQ ID NO: 218 - protein, 9 aa, synthetic construct
VLTGNLQTL

SEQ ID NO: 219 - protein, 9 aa, synthetic construct
ILRKATKRL

SEQ ID NO: 220 - protein, 9 aa, synthetic construct
RLNPMHQLL

SEQ ID NO: 221 - protein, 9 aa, synthetic construct
VLGKDAGAL

SEQ ID NO: 222 - protein, 9 aa, synthetic construct
SVYIEVLHL

SEQ ID NO: 223 - protein, 9 aa, synthetic construct
SVKREEEVL

SEQ ID NO: 224 - protein, 9 aa, synthetic construct
FVNRANQRL

SEQ ID NO: 225 - protein, 9 aa, synthetic construct
NVMGMIGIL

SEQ ID NO: 226 - protein, 9 aa, synthetic construct
VVVSIDRFL

SEQ ID NO: 227 - protein, 9 aa, synthetic construct
AVLRGFLIL

SEQ ID NO: 228 - protein, 9 aa, synthetic construct
APLMVAYML

SEQ ID NO: 229 - protein, 9 aa, synthetic construct
GPALSINEL

SEQ ID NO: 230 - protein, 9 aa, synthetic construct
TSSVYIEVL

SEQ ID NO: 231 - protein, 9 aa, synthetic construct
VSADPLASL

SEQ ID NO: 232 - protein, 9 aa, synthetic construct
QSRMQFSSL

SEQ ID NO: 233 - protein, 9 aa, synthetic construct
ESAVLRGFL

SEQ ID NO: 234 - protein, 9 aa, synthetic construct
LSINELSNL

SEQ ID NO: 235 - protein, 9 aa, synthetic construct
GGVRMVDIL

SEQ ID NO: 236 - protein, 9 aa, synthetic construct
NLQTLKLRV

SEQ ID NO: 237 - protein, 9 aa, synthetic construct
VLFQQMRDV

SEQ ID NO: 238 - protein, 9 aa, synthetic construct
VLIGQGDVV

SEQ ID NO: 239 - protein, 9 aa, synthetic construct
RVMVSPLAV

SEQ ID NO: 240 - protein, 9 aa, synthetic construct
PVAGGTSSV

SEQ ID NO: 241 - protein, 9 aa, synthetic construct
AVRGDLNFV

SEQ ID NO: 242 - protein, 9 aa, synthetic construct
EAQDVIMEV

SEQ ID NO: 243 - protein, 9 aa, synthetic construct
CLLQSLQQI

SEQ ID NO: 244 - protein, 9 aa, synthetic construct
SLQQIESMI

SEQ ID NO: 245 - protein, 9 aa, synthetic construct
DLEGLYEAI

SEQ ID NO: 246 - protein, 9 aa, synthetic construct
GVTRREVHI

SEQ ID NO: 247 - protein, 9 aa, synthetic construct
QVLSELQDI

SEQ ID NO: 248 - protein, 9 aa, synthetic construct
DPSHEGEGI

SEQ ID NO: 249 - protein, 9 aa, synthetic construct
EPRSLSCWI

SEQ ID NO: 250 - protein, 9 aa, synthetic construct
KSEKTHIHI

SEQ ID NO: 251 - protein, 9 aa, synthetic construct
QSERGEETI

SEQ ID NO: 252 - protein, 9 aa, synthetic construct
MSKEVNARI

SEQ ID NO: 253 - protein, 9 aa, synthetic construct
SSWVELDEI

SEQ ID NO: 254 - protein, 9 aa, synthetic construct
DGFEPNGCI

SEQ ID NO: 255 - protein, 9 aa, synthetic construct
EGIPLYDAI

SEQ ID NO: 256 - protein, 9 aa, synthetic construct
FGWKEPNII

SEQ ID NO: 257 - protein, 9 aa, synthetic construct
FLLMDALKL

SEQ ID NO: 258 - protein, 9 aa, synthetic construct
PGTFDLEGL

SEQ ID NO: 259 - protein, 9 aa, synthetic construct
ESSVKEKDL

SEQ ID NO: 260 - protein, 9 aa, synthetic construct
VLEIGDMLL

SEQ ID NO: 261 - protein, 9 aa, synthetic construct
KLLLIVQAL

SEQ ID NO: 262 - protein, 9 aa, synthetic construct
NLEPGTFDL

SEQ ID NO: 263 - protein, 9 aa, synthetic construct
CLINDPWVL

SEQ ID NO: 264 - protein, 9 aa, synthetic construct
GVYINTALL

SEQ ID NO: 265 - protein, 9 aa, synthetic construct
LSDNEGRLI

SEQ ID NO: 266 - protein, 9 aa, synthetic construct
TSDMRTEII

SEQ ID NO: 267 - protein, 9 aa, synthetic construct
EGRLIQNSI

SEQ ID NO: 268 - protein, 9 aa, synthetic construct
DGKWVRELI

SEQ ID NO: 269 - protein, 9 aa, synthetic construct
IGTMVMELI

SEQ ID NO: 270 - protein, 9 aa, synthetic construct
NATEIRASV

SEQ ID NO: 271 - protein, 9 aa, synthetic construct
SALILRGSV

SEQ ID NO: 272 - protein, 9 aa, synthetic construct
PACVYGLAV

SEQ ID NO: 273 - protein, 9 aa, synthetic construct
SAAFEDLRV

SEQ ID NO: 274 - protein, 9 aa, synthetic construct
DPKKTGGPI

SEQ ID NO: 275 - protein, 9 aa, synthetic construct
VSGIGRFYI

SEQ ID NO: 276 - protein, 9 aa, synthetic construct
NPAHKSQLV

SEQ ID NO: 277 - protein, 9 aa, synthetic construct
NSITIERMV

SEQ ID NO: 278 - protein, 9 aa, synthetic construct
RASAGQISV

SEQ ID NO: 279 - protein, 9 aa, synthetic construct
IAIGSVSLI

SEQ ID NO: 280 - protein, 9 aa, synthetic construct
RVWWTSNSI

SEQ ID NO: 281 - protein, 9 aa, synthetic construct
NPNQKIIAI

SEQ ID NO: 282 - protein, 9 aa, synthetic construct
APFSKDNSI

SEQ ID NO: 283 - protein, 9 aa, synthetic construct
GSVSLIIAI

SEQ ID NO: 284 - protein, 9 aa, synthetic construct
NSTIIEREI

SEQ ID NO: 285 - protein, 9 aa, synthetic construct
GSNRPILYI

SEQ ID NO: 286 - protein, 9 aa, synthetic construct
KSQVNRQVI

SEQ ID NO: 287 - protein, 9 aa, synthetic construct
FSVEGKKCI

SEQ ID NO: 288 - protein, 9 aa, synthetic construct
GSWPDGANI

SEQ ID NO: 289 - protein, 9 aa, synthetic construct
WAFDDGNDV

SEQ ID NO: 290 - protein, 9 aa, synthetic construct
GANINFMPV

SEQ ID NO: 291 - protein, 9 aa, synthetic construct
FSKDNSIRL

SEQ ID NO: 292 - protein, 9 aa, synthetic construct
LVCATCEQI

SEQ ID NO: 293 - protein, 9 aa, synthetic construct
PSGPLKAEI

SEQ ID NO: 294 - protein, 9 aa, synthetic construct
LAKGILGFV

SEQ ID NO: 295 - protein, 9 aa, synthetic construct
TAEGALGLV

SEQ ID NO: 296 - protein, 9 aa, synthetic construct
VASQARQMV

SEQ ID NO: 297 - protein, 9 aa, synthetic construct
LSIIPSGPL

SEQ ID NO: 298 - protein, 9 aa, synthetic construct
RGLQRRRFV

SEQ ID NO: 299 - protein, 9 aa, synthetic construct
KAVKLYKKL

SEQ ID NO: 300 - protein, 9 aa, synthetic construct
GALASCMGL

SEQ ID NO: 301 - protein, 9 aa, synthetic construct
GILGFVFTL

SEQ ID NO: 302 - protein, 9 aa, synthetic construct
ALSYSTGAL

SEQ ID NO: 303 - protein, 9 aa, synthetic construct
LSPLAKGIL

SEQ ID NO: 304 - protein, 9 aa, synthetic construct
CSGSSDPLV

SEQ ID NO: 305 - protein, 9 aa, synthetic construct
IAANIIGIL

SEQ ID NO: 306 - protein, 9 aa, synthetic construct
IIGILHLIL

SEQ ID NO: 307 - protein, 9 aa, synthetic construct
YIVERPSAV

SEQ ID NO: 308 - protein, 9 aa, synthetic construct
GIKSLKLAV

SEQ ID NO: 309 - protein, 9 aa, synthetic construct
AIDKITSKV

SEQ ID NO: 310 - protein, 9 aa, synthetic construct
IIDHEFSEV

SEQ ID NO: 311 - protein, 9 aa, synthetic construct
KILTIYSTV

SEQ ID NO: 312 - protein, 9 aa, synthetic construct
SLITILLVV

SEQ ID NO: 313 - protein, 9 aa, synthetic construct
TLTENNVPV

SEQ ID NO: 314 - protein, 9 aa, synthetic construct
PLIGPRPLV

SEQ ID NO: 315 - protein, 9 aa, synthetic construct
DLKRGSCTV

SEQ ID NO: 316 - protein, 9 aa, synthetic construct
KLAVGLRNV

SEQ ID NO: 317 - protein, 9 aa, synthetic construct
TLDEHDANV

SEQ ID NO: 318 - protein, 9 aa, synthetic construct
NVNNLYNKV

SEQ ID NO: 319 - protein, 9 aa, synthetic construct
ASLITILLV

SEQ ID NO: 320 - protein, 9 aa, synthetic construct
QSTNSTETV

SEQ ID NO: 321 - protein, 9 aa, synthetic construct
YSTVASSLV

SEQ ID NO: 322 - protein, 9 aa, synthetic construct
AATALANTI

SEQ ID NO: 323 - protein, 9 aa, synthetic construct
TANESGRLI

SEQ ID NO: 324 - protein, 9 aa, synthetic construct
EAMVSRARI

SEQ ID NO: 325 - protein, 9 aa, synthetic construct
AIATPGMQI

SEQ ID NO: 326 - protein, 9 aa, synthetic construct
WIPKRNRSI

SEQ ID NO: 327 - protein, 9 aa, synthetic construct
RIDFESGRI

SEQ ID NO: 328 - protein, 9 aa, synthetic construct
EIMKICSTI

SEQ ID NO: 329 - protein, 9 aa, synthetic construct
FLEESHPGI

SEQ ID NO: 330 - protein, 9 aa, synthetic construct
RLNKRSYLI

SEQ ID NO: 331 - protein, 9 aa, synthetic construct
ILNTSQRGI

SEQ ID NO: 332 - protein, 9 aa, synthetic construct
FVEALARSI

SEQ ID NO: 333 - protein, 9 aa, synthetic construct
AVATTHSWI

SEQ ID NO: 334 - protein, 9 aa, synthetic construct
NPRMFLAMI

SEQ ID NO: 335 - protein, 9 aa, synthetic construct
EIDSVNNAV

SEQ ID NO: 336 - protein, 9 aa, synthetic construct
SSDDFALIV

SEQ ID NO: 337 - protein, 9 aa, synthetic construct
ESADMSIGV

SEQ ID NO: 338 - protein, 9 aa, synthetic construct
VSHKEIDSV

SEQ ID NO: 339 - protein, 9 aa, synthetic construct
PSSSYRRPV

SEQ ID NO: 340 - protein, 9 aa, synthetic construct
TATREGKHI

SEQ ID NO: 341 - protein, 9 aa, synthetic construct
RAFTDEGAI

SEQ ID NO: 342 - protein, 9 aa, synthetic construct
TLKANFSVI

SEQ ID NO: 343 - protein, 9 aa, synthetic construct
RSSTLGLDI

SEQ ID NO: 344 - protein, 9 aa, synthetic construct
SIRLMDRCL

SEQ ID NO: 345 - protein, 9 aa, synthetic construct
CIRMDQAIV

SEQ ID NO: 346 - protein, 9 aa, synthetic construct
QVDCFLWHV

SEQ ID NO: 347 - protein, 9 aa, synthetic construct
LPGHTDKDV

SEQ ID NO: 348 - protein, 9 aa, synthetic construct
AIVDKNITL

SEQ ID NO: 349 - protein, 9 aa, synthetic construct
EGKHIVERI

SEQ ID NO: 350 - protein, 9 aa, synthetic construct
SSEDLNGMI

SEQ ID NO: 351 - protein, 9 aa, synthetic construct
QALQLLLEV

SEQ ID NO: 352 - protein, 9 aa, synthetic construct
EIRWLIEEV

SEQ ID NO: 353 - protein, 9 aa, synthetic construct
KLFSKQEWI

SEQ ID NO: 354 - protein, 9 aa, synthetic construct
RVGMHKRIV

SEQ ID NO: 355 - protein, 9 aa, synthetic construct
GVRVSKMGV

SEQ ID NO: 356 - protein, 9 aa, synthetic construct
RVRDQRGNV

SEQ ID NO: 357 - protein, 9 aa, synthetic construct
NVLLSPEEV

SEQ ID NO: 358 - protein, 9 aa, synthetic construct
DVLGTFDTV

SEQ ID NO: 359 - protein, 9 aa, synthetic construct
IVVRGNSPV

SEQ ID NO: 360 - protein, 9 aa, synthetic construct
NVLIGQGDV

SEQ ID NO: 361 - protein, 9 aa, synthetic construct
GPVHFRNQV

SEQ ID NO: 362 - protein, 9 aa, synthetic construct
DPDEGTAGV

SEQ ID NO: 363 - protein, 9 aa, synthetic construct
TSTVHYPKV

SEQ ID NO: 364 - protein, 9 aa, synthetic construct
LSAKEAQDV

SEQ ID NO: 365 - protein, 9 aa, synthetic construct
SSVKREEEV

SEQ ID NO: 366 - protein, 9 aa, synthetic construct
QSIAEAIIV

SEQ ID NO: 367 - protein, 9 aa, synthetic construct
YSSTERVVV

SEQ ID NO: 368 - protein, 9 aa, synthetic construct
VSIDRFLRV

SEQ ID NO: 369 - protein, 9 aa, synthetic construct
GGEVRNDDV

SEQ ID NO: 370 - protein, 9 aa, synthetic construct
EGYEEFTMV

SEQ ID NO: 371 - protein, 9 aa, synthetic construct
WGIEPIDNV

SEQ ID NO: 372 - protein, 9 aa, synthetic construct
RGSGMRIVV

SEQ ID NO: 373 - protein, 9 aa, synthetic construct
IGQGDVVLV

SEQ ID NO: 374 - protein, 9 aa, synthetic construct
VAYMLEREL

SEQ ID NO: 375 - protein, 9 aa, synthetic construct
TGNLQTLKL

SEQ ID NO: 376 - protein, 9 aa, synthetic construct
LAKGEKANV

SEQ ID NO: 377 - protein, 9 aa, synthetic construct
RATVSADPL

SEQ ID NO: 378 - protein, 9 aa, synthetic construct
SADPLASLL

SEQ ID NO: 379 - protein, 9 aa, synthetic construct
LLRTAVGQV

SEQ ID NO: 380 - protein, 9 aa, synthetic construct
ASICTHLEV

SEQ ID NO: 381 - protein, 9 aa, synthetic construct
NSICNTTGV

SEQ ID NO: 382 - protein, 9 aa, synthetic construct
VSRPMFLYV

SEQ ID NO: 383 - protein, 9 aa, synthetic construct
ESRKLLLIV

SEQ ID NO: 384 - protein, 9 aa, synthetic construct
MATKADYTL

SEQ ID NO: 385 - protein, 9 aa, synthetic construct
CAAMDDFQL

SEQ ID NO: 386 - protein, 9 aa, synthetic construct
LAKSVFNSL

SEQ ID NO: 387 - protein, 9 aa, synthetic construct
SAESRKLLL

SEQ ID NO: 388 - protein, 9 aa, synthetic construct
NASWFNSFL

SEQ ID NO: 389 - protein, 9 aa, synthetic construct
EITGTVRRL

SEQ ID NO: 390 - protein, 9 aa, synthetic construct
FIIKGRSHL

SEQ ID NO: 391 - protein, 9 aa, synthetic construct
SIGKVCRTL

SEQ ID NO: 392 - protein, 9 aa, synthetic construct
LINDPWVLL

SEQ ID NO: 393 - protein, 9 aa, synthetic construct
SLPPNFSSL

SEQ ID NO: 394 - protein, 9 aa, synthetic construct
FLKTTPRPL

SEQ ID NO: 395 - protein, 9 aa, synthetic construct
NSLYASPQL

SEQ ID NO: 396 - protein, 9 aa, synthetic construct
FSAESRKLL

SEQ ID NO: 397 - protein, 9 aa, synthetic construct
TGVEKPKFL

SEQ ID NO: 398 - protein, 9 aa, synthetic construct
KGINPNYLL

SEQ ID NO: 399 - protein, 9 aa, synthetic construct
KGVYINTAL

SEQ ID NO: 400 - protein, 9 aa, synthetic construct
YLLTWKQVL

SEQ ID NO: 401 - protein, 9 aa, synthetic construct
SITIERMVL

SEQ ID NO: 402 - protein, 9 aa, synthetic construct
VGIDPFRLL

SEQ ID NO: 403 - protein, 9 aa, synthetic construct
GIGTMVMEL

SEQ ID NO: 404 - protein, 9 aa, synthetic construct
LIFLARSAL

SEQ ID NO: 405 - protein, 9 aa, synthetic construct
KLSDNEGRL

SEQ ID NO: 406 - protein, 9 aa, synthetic construct
HLMIWHSNL

SEQ ID NO: 407 - protein, 9 aa, synthetic construct
FLARSALIL

SEQ ID NO: 408 - protein, 9 aa, synthetic construct
CLPACVYGL

SEQ ID NO: 409 - protein, 9 aa, synthetic construct
LVGIDPFRL

SEQ ID NO: 410 - protein, 9 aa, synthetic construct
CSLMQGSTL

SEQ ID NO: 411 - protein, 9 aa, synthetic construct
GSVAHKSCL

SEQ ID NO: 412 - protein, 9 aa, synthetic construct
NGEDATAGL

SEQ ID NO: 413 - protein, 9 aa, synthetic construct
PGNAEIEDL

SEQ ID NO: 414 - protein, 9 aa, synthetic construct
KADTRVLFI

SEQ ID NO: 415 - protein, 9 aa, synthetic construct
AIGSVSLII

SEQ ID NO: 416 - protein, 9 aa, synthetic construct
AIICLLMQI

SEQ ID NO: 417 - protein, 9 aa, synthetic construct
SIGSWSKNI

SEQ ID NO: 418 - protein, 9 aa, synthetic construct
YINMADYSI

SEQ ID NO: 419 - protein, 9 aa, synthetic construct
HLGTKQVCI

SEQ ID NO: 420 - protein, 9 aa, synthetic construct
VLFIREGKI

SEQ ID NO: 421 - protein, 9 aa, synthetic construct
SVSLIIAII

SEQ ID NO: 422 - protein, 9 aa, synthetic construct
QVMPCEPII

SEQ ID NO: 423 - protein, 9 aa, synthetic construct
TVHLNSTII

SEQ ID NO: 424 - protein, 9 aa, synthetic construct
ILERNTVHL

SEQ ID NO: 425 - protein, 9 aa, synthetic construct
IAIGSVSLI

SEQ ID NO: 426 - protein, 9 aa, synthetic construct
ASIIYDGML

SEQ ID NO: 427 - protein, 9 aa, synthetic construct
ESSYVCSGL

SEQ ID NO: 428 - protein, 9 aa, synthetic construct
IGSWSKNIL

SEQ ID NO: 429 - protein, 9 aa, synthetic construct
SGKADTRVL

SEQ ID NO: 430 - protein, 9 aa, synthetic construct
PLAGSAQHV

SEQ ID NO: 431 - protein, 9 aa, synthetic construct
CSNPTNNQV

SEQ ID NO: 432 - protein, 9 aa, synthetic construct
LSAGGDIWV

SEQ ID NO: 433 - protein, 9 aa, synthetic construct
PIILERNTV

SEQ ID NO: 434 - protein, 9 aa, synthetic construct
CINGTCAVV

SEQ ID NO: 435 - protein, 9 aa, synthetic construct
TLLMNELGV

SEQ ID NO: 436 - protein, 9 aa, synthetic construct
CLLMQIAIL

SEQ ID NO: 437 - protein, 9 aa, synthetic construct
QAYQNRMGV

SEQ ID NO: 438 - protein, 9 aa, synthetic construct
EIAQRLEDV

SEQ ID NO: 439 - protein, 9 aa, synthetic construct
LIYNRMGTV

SEQ ID NO: 440 - protein, 9 aa, synthetic construct
LIRHENRMV

SEQ ID NO: 441 - protein, 9 aa, synthetic construct
LLTEVETYV

SEQ ID NO: 442 - protein, 9 aa, synthetic construct
SSTGLKDDL

SEQ ID NO: 443 - protein, 9 aa, synthetic construct
LILWILDRL

SEQ ID NO: 444 - protein, 9 aa, synthetic construct
ILHLILWIL

SEQ ID NO: 445 - protein, 9 aa, synthetic construct
DGHFVNIEL

SEQ ID NO: 446 - protein, 9 aa, synthetic construct
TASLITILL

SEQ ID NO: 447 - protein, 9 aa, synthetic construct
CATSLGHPL

SEQ ID NO: 448 - protein, 9 aa, synthetic construct
IAPWYGHIL

SEQ ID NO: 449 - protein, 9 aa, synthetic construct
IAMGFAAFL

SEQ ID NO: 450 - protein, 9 aa, synthetic construct
EINRTFKPL

SEQ ID NO: 451 - protein, 9 aa, synthetic construct
RINYYWSVL

SEQ ID NO: 452 - protein, 9 aa, synthetic construct
YIGIKSLKL

SEQ ID NO: 453 - protein, 9 aa, synthetic construct
FIEGGWSGL

SEQ ID NO: 454 - protein, 9 aa, synthetic construct
DIWAYNAEL

SEQ ID NO: 455 - protein, 9 aa, synthetic construct
VLLENQKTL

SEQ ID NO: 456 - protein, 9 aa, synthetic construct
PVTHAKELL

SEQ ID NO: 457 - protein, 9 aa, synthetic construct
SVLKPGQTL

SEQ ID NO: 458 - protein, 9 aa, synthetic construct
VPVTHAKEL

SEQ ID NO: 459 - protein, 9 aa, synthetic construct
GGREWSYIV

SEQ ID NO: 460 - protein, 9 aa, synthetic construct
NGLCYPGNV

SEQ ID NO: 461 - protein, 9 aa, synthetic construct
VPAEMLANI

SEQ ID NO: 462 - protein, 9 aa, synthetic construct
LPSFGVSGI

SEQ ID NO: 463 - protein, 9 aa, synthetic construct
NPFVSHKEI

SEQ ID NO: 464 - protein, 9 aa, synthetic construct
NSCLETMEI

SEQ ID NO: 465 - protein, 9 aa, synthetic construct
LSTVLGVSI

SEQ ID NO: 466 - protein, 9 aa, synthetic construct
QSSDDFALI

SEQ ID NO: 467 - protein, 9 aa, synthetic construct
SSYRRPVGI

SEQ ID NO: 468 - protein, 9 aa, synthetic construct
TGAPQLNPI

SEQ ID NO: 469 - protein, 9 aa, synthetic construct
RGKLKRRAI

SEQ ID NO: 470 - protein, 9 aa, synthetic construct
DGGPNLYNI

SEQ ID NO: 471 - protein, 9 aa, synthetic construct
TALANTIEV

SEQ ID NO: 472 - protein, 9 aa, synthetic construct
LIDFLKDVV

SEQ ID NO: 473 - protein, 9 aa, synthetic construct
QIRGFVYFV

SEQ ID NO: 474 - protein, 9 aa, synthetic construct
TIGKKKQRL

SEQ ID NO: 475 - protein, 9 aa, synthetic construct
ISSMVEAMV

SEQ ID NO: 476 - protein, 9 aa, synthetic construct
SGYAQTDCV

SEQ ID NO: 477 - protein, 9 aa, synthetic construct
PGMQIRGFV

SEQ ID NO: 478 - protein, 9 aa, synthetic construct
LARSICEKL

SEQ ID NO: 479 - protein, 9 aa, synthetic construct
VIFNRLEAL

SEQ ID NO: 480 - protein, 9 aa, synthetic construct
AIVGEISPL

SEQ ID NO: 481 - protein, 9 aa, synthetic construct
RLRRDQKSL

SEQ ID NO: 482 - protein, 9 aa, synthetic construct
SLRGRSSTL

SEQ ID NO: 483 - protein, 9 aa, synthetic construct
DVKNAIEVL

SEQ ID NO: 484 - protein, 9 aa, synthetic construct
SSFQVDCFL

SEQ ID NO: 485 - protein, 9 aa, synthetic construct
ASVPAPRYL

SEQ ID NO: 486 - protein, 9 aa, synthetic construct
RGRSSTLGL

SEQ ID NO: 487 - protein, 9 aa, synthetic construct
QLSQKFEEI

SEQ ID NO: 488 - protein, 9 aa, synthetic construct
LLLEVEQEI

SEQ ID NO: 489 - protein, 9 aa, synthetic construct
TVSSFQDIL

SEQ ID NO: 490 - protein, 9 aa, synthetic construct
EAAMRMGDL

SEQ ID NO: 491 - protein, 9 aa, synthetic construct
LIEEVRHRL

SEQ ID NO: 492 - protein, 9 aa, synthetic construct
EIRTFSFQL